(19) United States
(12) Patent Application Publication
(10) Pub. No.: US 2025/0134412 A1
(43) Pub. Date: May 1, 2025

(54) INTERPOLATION DEVICE, GAIT MEASUREMENT SYSTEM, INTERPOLATION METHOD, AND RECORDING MEDIUM

(71) Applicant: NEC Corporation, Minato-ku, Tokyo (JP)

(72) Inventors: Chenhui HUANG, Tokyo (JP); Zhenwei WANG, Tokyo (JP)

(73) Assignee: NEC Corporation, Minato-ku, Tokyo (JP)

(21) Appl. No.: 18/570,188

(22) PCT Filed: Jun. 21, 2021
(86) PCT No.: PCT/JP2021/023444

§ 371 Date: Dec. 14, 2023

(51) Int. Cl. A61B 5/11; A61B 5/00

(52) U.S. Cl. CPC A61B 5/112; A61B 5/7246

(57) ABSTRACT

An interpolation device includes a gait information processing unit that generates a gait waveform for each gait cycle by using time-series data of sensor data regarding a movement of a foot and specifies a missing section of data in the time-series data, a missing information processing unit that calculates a gait phase of the specified missing section, and an interpolation unit that generates interpolation data for interpolating the missing section by using data of the gait phase of the missing section in a gait waveform with a gait cycle different from a gait cycle including the missing section, and interpolates the generated interpolation data to the missing section.

### TECHNICAL FIELD

The present disclosure relates to an interpolation device or the like that interpolates a missing of time-series data.

### BACKGROUND ART

With increasing interest in healthcare that performs physical condition management, a service that measures features included in a walking pattern (also referred to as gait) and provides information corresponding to the gait to a user has attracted attention. For example, a measurement device including an inertial sensor is mounted on footwear such as shoes, and a device for analyzing a gait of a user has been developed. In such gait analysis, when data measured by a sensor (also referred to as sensor data) is wirelessly transmitted from a measurement device, data missing may occur due to a factor such as a communication failure.

PTL 1 discloses a gait measurement system for the purpose of removing a drift exhibiting different characteristics between a period (stance phase) in which a foot is in contact with the ground and a period (swing phase) in which the foot is away from the ground. The system of PTL 1 detects at least one gait phase from acceleration data measured by an inertial measurement unit, and calculates speed data by time-integrating the acceleration data. The system of PTL 1 calculates a correction amount relevant to each gait phase based on the gait phase and the speed data. The system of PTL 1 calculates corrected speed data by subtracting the correction amount from the speed data relevant to each gait phase, and calculates trajectory data by time-integrating the calculated corrected speed data.

PTL 2 discloses a signal interpolation method for interpolating a missing part of an input signal. In the method of PTL 2, the cycle of the signal before the missing of the input signal is detected, and the detected input signal is accumulated. In the method of PTL 2, the accumulated signal is read according to the detection of the input signal, and the missing part is interpolated. In the method of PTL 2, the polarity and the level at the time of starting the missing of the input signal are identified, and the accumulated waveform signals of the polarity and the level maintaining continuity with the input signal immediately before the missing are sequentially read. In the method of PTL 2, the level of the accumulated waveform signal is gradually approximated to the level of the input signal, whereby the missing part of the input signal is interpolated so as to maintain continuity.

PTL 3 discloses a data interpolation device that interpolates data missing measured by a sensor or a device connected to a network. The device of PTL 3 stores a time-series model of data and an interpolation method for interpolating missing of data. The interpolation method is relevant to a phase specified as a range in which temporal changes of data are common in the time-series model. The device of PTL 3 accumulates data output from an external device for each external device, and determines which time-series model the temporal change pattern of the accumulated data corresponds to. The device of PTL 3 determines in which phase of the determined time-series model the missing included in a part of the accumulated data is located, and interpolates the missing by an interpolation method corresponding to the determined phase.

PTL 4 discloses a biological information measurement device that complements a missing part included in biological information. The device of PTL 4 complements a missing part included in time-series data (biological information) measured by the pulse wave sensor and the electrocardiogram sensor. The device of PTL 4 complements a missing part of data measured by one sensor among time-series data measured by two sensors based on data measured by the other sensor. The device of PTL 4 specifies a relevant section in which an index indicating a correlation with time-series data in a section at the same time as the missing part satisfies a predetermined condition among data before a start point of the missing part of the time-series data measured by the other sensor. The device of PTL 4 complements a missing part by using time-series data of a section at the same time as the relevant section in data measured by one sensor.

### CITATION LIST

### Patent Literature

PTL 1: WO 2020/105115 A1
PTL 2: JP 61-227432 A
PTL 3: JP 2014-102779 A
PTL 4: JP 2010-264168 A

### SUMMARY OF INVENTION

### Technical Problem

According to the method of PTL 1, it is possible to remove drifts exhibiting clearly different characteristics in different gait periods, such as a stance phase and a swing phase. However, in the method of PTL 1, it is not possible to interpolate a missing part locally generated in each gait period such as a stance phase and a swing phase. Therefore, in the method of PTL 1, it is not possible to restore the characteristics included in the missing part.

In the method of PTL 2, when there is a missing in the input signal, the accumulated signal is repeatedly read to interpolate the missing part so that continuity is obtained from the missing start time to the missing end time. According to the method of PTL 2, in a case where the periodicity and the shape of the input signal are simple, missing parts can be interpolated. However, in the method of PTL 2, in a case where regularity or a waveform is complicated as in sensor data, it is difficult to interpolate a missing part so that continuity can be obtained from a missing start time to a missing end time. Therefore, in the method of PTL 2, there is a possibility that features included in the missing part are lost.

In the method of PTL 3, it is necessary to store the time-series model and the interpolation method in advance. Therefore, in the method of PTL 3, it is not possible to interpolate a data missing for a missing for which the time-series model and the interpolation method cannot be specified.

In the method of PTL 4, two pieces of time-series data measured at the same time and correlated with each other are associated with each other, and a missing part of one piece of time-series data is complemented based on the other piece of data. Therefore, the method of PTL 4 cannot complement the missing part unless there is time-series data measured at the same time.

An object of the present disclosure is to provide an interpolation device or the like capable of interpolating data of a missing section including a feature of the missing section of data included in time-series data of sensor data.

### Solution to Problem

An interpolation device according to an aspect of the present disclosure includes a gait information processing unit that generates a gait waveform for each gait cycle by using time-series data of sensor data regarding a movement of a foot and specifies a missing section of data in the time-series data, a missing information processing unit that calculates a gait phase of the specified missing section, and an interpolation unit that generates interpolation data for interpolating the missing section by using data of the gait phase of the missing section in a gait waveform with a gait cycle different from a gait cycle including the missing section, and interpolates the generated interpolation data to the missing section.

In an interpolation method according to an aspect of the present disclosure, a computer executes generating a gait waveform for each gait cycle by using time-series data of sensor data regarding a movement of a foot, specifying a missing section of data in the time-series data, calculating a gait phase of the specified missing section, generating interpolation data for interpolating the missing section by using data of the gait phase of the missing section in a gait waveform having a gait cycle different from a gait cycle including the missing section, and interpolating the generated interpolation data into the missing section.

A program according to an aspect of the present disclosure causes a computer to execute: generating a gait waveform for each gait cycle by using time-series data of sensor data regarding a movement of a foot, specifying a missing section of data in the time-series data, calculating a gait phase of the specified missing section, generating interpolation data for interpolating the missing section by using data of the gait phase of the missing section in a gait waveform having a gait cycle different from a gait cycle including the missing section, and interpolating the generated interpolation data into the missing section.

### Advantageous Effects of Invention

According to the present disclosure, it is possible to provide an interpolation device or the like capable of interpolating data in a missing section including a feature of a missing section of data included in time-series data of sensor data.

### BRIEF DESCRIPTION OF DRAWINGS

FIG. 1 is a block diagram illustrating an example of a configuration of a gait measurement system according to a first example embodiment.

FIG. 2 is a conceptual diagram illustrating an arrangement example of a measurement device of the gait measurement system according to the first example embodiment.

FIG. 3 is a conceptual diagram for explaining a coordinate system set in the measurement device of the gait measurement system according to the first example embodiment.

FIG. 4 is a conceptual diagram for explaining a gait cycle.

FIG. 5 is a conceptual diagram for explaining a missing occurring in time-series data of sensor data.

FIG. 6 is a conceptual diagram for explaining an example of interpolating a missing occurring in time-series data of sensor data by an analytical method.

FIG. 7 is a conceptual diagram for explaining interpolation of a missing section by an interpolation device of the gait measurement system according to the first example embodiment.

FIG. 8 is a block diagram illustrating an example of a configuration of the measurement device of the gait measurement system according to the first example embodiment.

FIG. 9 is a block diagram illustrating an example of a configuration of the interpolation device of the gait measurement system according to the first example embodiment.

FIG. 10 is a conceptual diagram illustrating an example of missing interpolation by the interpolation device of the gait measurement system according to the first example embodiment.

FIG. 11 is a conceptual diagram for explaining missing interpolation by the interpolation device of the gait measurement system according to the first example embodiment.

FIG. 12 is a block diagram illustrating an example of a configuration of a gait measurement device of the gait measurement system according to the first example embodiment.

FIG. 13 is a flowchart for explaining an example of the operation of a gait information processing unit included in the interpolation device of the gait measurement device of the gait measurement system according to the first example embodiment.

FIG. 14 is a flowchart for explaining an example of the operation of a missing information processing unit included in the interpolation device of the gait measurement device of the gait measurement system according to the first example embodiment.

FIG. 15 is a flowchart for explaining an example of the operation of an interpolation unit included in the interpolation device of the gait measurement device of the gait measurement system according to the first example embodiment.

FIG. 16 is a flowchart for explaining another example of the operation of the interpolation unit included in the interpolation device of the gait measurement device of the gait measurement system according to the first example embodiment.

FIG. 17 is a block diagram illustrating an example of a configuration of a gait measurement system according to a second example embodiment.

FIG. 18 is a block diagram illustrating an example of a configuration of an interpolation device of the gait measurement system according to the second example embodiment.

FIG. 19 is an example of time-series data of sensor data over a plurality of gait cycles.

FIG. 20 is an example of a gait waveform obtained by normalizing time-series data for one gait cycle extracted from time-series data of sensor data over a plurality of gait cycles.

FIG. 21 is a frequency distribution of data values in a gait phase P1 and a gait phase P2 in which gait waveforms of a plurality of gait cycles are set as samples (whole).

FIG. 22 is a graph illustrating a correlation of deviation values in an overall distribution of data values of data N and data N+1 in a continuous gait phase.

FIG. 23 is a graph illustrating a correlation coefficient of a deviation value according to a distance from a target gait phase.

FIG. 24 is a graph for explaining interpolation of missing sections by the interpolation device according to the second example embodiment.

FIG. 25 is a flowchart for explaining the operation of the interpolation device according to the second example embodiment.

FIG. 26 is a block diagram illustrating an example of a configuration of an interpolation device according to a third example embodiment.

FIG. 27 is a block diagram illustrating an example of a hardware configuration that executes control and processing according to each example embodiment.

### EXAMPLE EMBODIMENT

Hereinafter, example embodiments of the present invention will be described with reference to the drawings. However, the example embodiments described below may be technically limited for carrying out the present invention, but the scope of the invention is not limited to the following. In all the drawings used in the following description of the example embodiment, the same reference numerals are given to the same parts unless there is a particular reason. In the following example embodiments, repeated description of similar configurations and operations may be omitted.

### First Example Embodiment

First, a gait measurement system according to a first example embodiment will be described with reference to the drawings. The gait measurement system according to the present example embodiment measures a physical quantity (sensor data) regarding a motion of a foot by a measurement device installed on footwear worn by a user. The measurement device includes an acceleration sensor and an angular velocity sensor. For example, the physical quantity regarding the movement of the foot includes accelerations in three axial directions (also referred to as spatial accelerations) measured by the acceleration sensor and angular velocities around three axes (also referred to as spatial angular velocities) measured by the angular velocity sensor. The gait measurement system according to the present example embodiment interpolates a missing portion of data generated at the time of communication of the measured sensor data or the like.

### (Configuration)

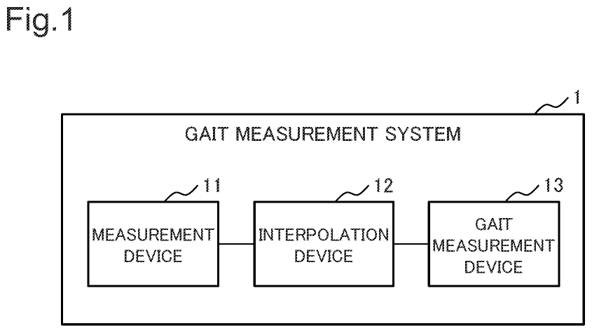

FIG. 1 is a block diagram illustrating a configuration of a gait measurement system 1 according to the present example embodiment. The gait measurement system 1 includes a measurement device 11, an interpolation device 12, and a gait measurement device 13. The interpolation device 12 may be connected to the measurement device 11 and the gait measurement device 13 in a wired manner or in a wireless manner. The measurement device 11, the interpolation device 12, and the gait measurement device 13 may be configured by a single device. The gait measurement system 1 may include the interpolation device 12 and the gait measurement device 13 except for the measurement device 11.

The measurement device 11 is installed on the foot portion. For example, the measurement device 11 is installed in a footwear such as shoes. For example, the measurement device 11 is disposed at a position on the back side of the arch of foot. The measurement device 11 includes an acceleration sensor and an angular velocity sensor. The measurement device 11 measures acceleration measured by the acceleration sensor (also referred to as spatial acceleration) and an angular velocity measured by the angular velocity sensor (also referred to as spatial angular velocity) as physical quantities regarding the movement of the foot of the user wearing the footwear. The physical quantity regarding the movement of the foot measured by the measurement device 11 includes a speed, an angle, and a position (trajectory) calculated by integrating the acceleration and the angular velocity. The measurement device 11 converts the measured physical quantity into digital data (also referred to as sensor data). The measurement device 11 transmits the converted sensor data to the interpolation device 12. The sensor data includes a time stamp relevant to a time at which the sensor data is acquired. The time stamp is a time-series number assigned to the sensor data. For example, the measurement device 11 is connected to the interpolation device 12 via a mobile terminal (not illustrated) carried by the user.

A mobile terminal (not illustrated) is a communication device that can be carried by a user. For example, the mobile terminal is a portable communication device having a communication function, such as a smartphone, a smart watch, or a mobile phone. The mobile terminal receives, from the measurement device 11, sensor data regarding the movement of the foot of the user. The mobile terminal transmits the received sensor data to a server, a cloud, or the like on which the interpolation device 12 is mounted. The functions of the interpolation device 12 and the gait measurement device 13 may be achieved by application software or the like installed in the mobile terminal. In this case, the mobile terminal processes the received sensor data by application software or the like installed therein.

The measurement device 11 is achieved by, for example, an inertial measurement device including an acceleration sensor and an angular velocity sensor. An example of the inertial measurement device is an inertial measurement unit (IMU). The IMU includes an acceleration sensor that measures accelerations in three axial directions and an angular velocity sensor that measures angular velocities around three axes. The measurement device 11 may be achieved by an inertial measurement device such as a vertical gyro (VG) or an attitude heading (AHRS). The measurement device 11 may be achieved by a Global Positioning System/Inertial Navigation System (GPS/INS).

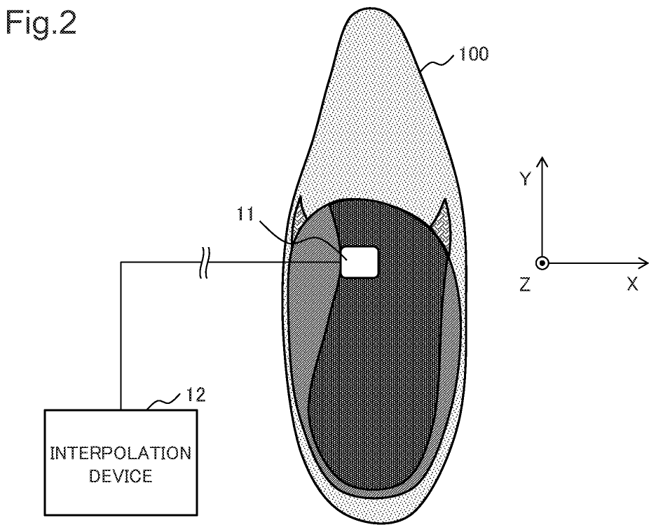

FIG. 2 is a conceptual diagram illustrating an example of disposed the measurement device 11 in a shoe 100. In the example of FIG. 2, the measurement device 11 is disposed at a position relevant to the back side of the arch of foot. For example, the measurement device 11 is disposed in an insole inserted into the shoe 100. For example, the measurement device 11 is disposed on the bottom surface of the shoe 100. The measurement device 11 may be embedded in the main body of the shoe 100. The measurement device 11 may be detachable from the shoe 100 or may not be detachable from the shoe 100. The measurement device 11 may be disposed at a position other than the back side of the arch of foot as long as the sensor data regarding the movement of the foot can be acquired. The measurement device 11 may be disposed on a sock worn by the user or a decorative article such as an anklet worn by the user. The measurement device 11 may be directly attached to the foot or may be embedded in the foot. FIG. 2 illustrates an example in which the measurement device 11 is disposed on the shoe 100 on the right foot side, but the measurement device 11 may be disposed on the shoe 100 for both feet. If the measurement device 11 is disposed on the shoe 100 for both feet, the gait can be measured based on the movement of the foot for both feet.

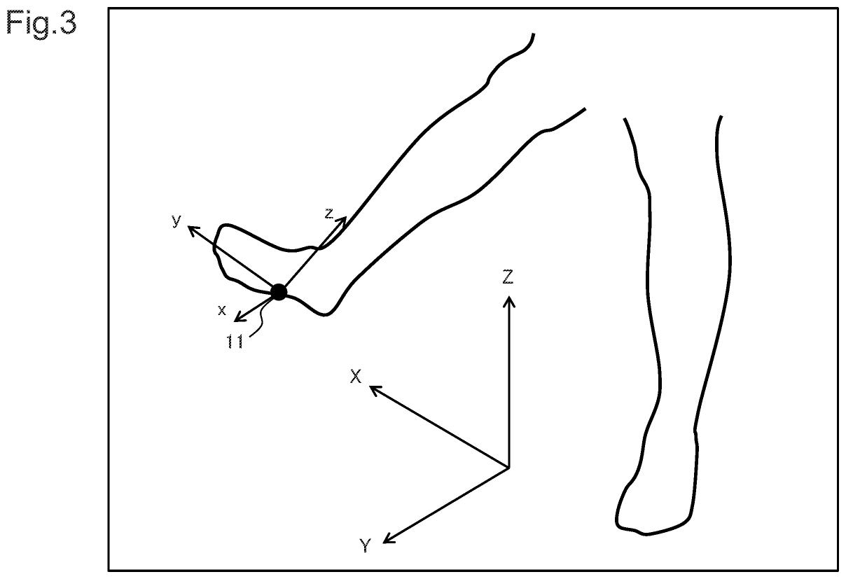

FIG. 3 is a conceptual diagram for explaining a local coordinate system (x axis, y axis, z axis) set in the measurement device 11 and a world coordinate system (X axis, Y axis, Z axis) set with respect to the ground in a case where the measurement device 11 is installed on the back side of the arch of feet. In the world coordinate system (X axis, Y axis, Z axis), in a state where the user is standing upright, a lateral direction of the user is set to an X-axis direction (rightward direction is positive), a front direction of the user (traveling direction) is set to a Y-axis direction (forward direction is positive), and a gravity direction is set to a Z-axis direction (vertically upward direction is positive). In the present example embodiment, the local coordinate system including an x direction, a y direction, and a z direction based on the measurement device 11 is set.

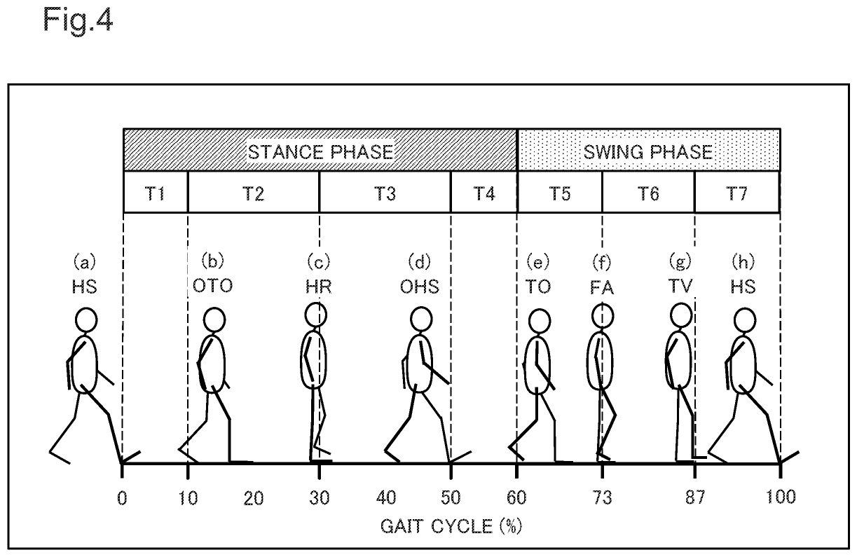

FIG. 4 is a conceptual diagram for explaining one gait cycle based on the right foot. FIG. 4 illustrates a gait cycle of the right foot with a time point at which the heel of the right foot lands on the ground as a start point and a time point at which the heel of the right foot next lands on the ground as an end point. FIG. 4 is a normalized gait cycle with one gait cycle of the right foot as 100%. The one gait cycle of one foot is roughly divided into a stance phase in which at least a part of the back side of the foot is in contact with the ground and a swing phase in which the back side of the foot is separated from the ground. In the present example embodiment, the gait cycle is normalized such that the stance phase occupies 60% and the swing phase occupies 40%. The stance phase is further subdivided into an initial stance period T1, a mid-stance period T2, a terminal stance period T3, and a pre-swing period T4. The swing phase is further subdivided into an initial swing period T5, a mid-swing period T6, and a terminal swing period T7. The gait waveform in one gait cycle may not have a time point when the heel lands on the ground as the start point. For example, the gait waveform for one gait cycle may have a start point and an end point at which the heel rises.

FIG. 4(a) represents an event in which the heel of the right foot lands the ground (HS: Heel Strike). FIG. 4(b) illustrates an event (opposite toe off) in which the toe of the left foot is separated from the ground while the sole of the right foot is in contact with the ground (OTO: Opposite Toe Off). FIG. 4(c) illustrates an event (heel rise) in which the heel of the right foot is lifted while the sole of the right foot is in contact with the ground (HR: Heel Rise). FIG. 4(d) is an event (opposite heel strike) in which the heel of the left foot is grounded (OHS: Opposite Heel Strike). FIG. 4(e) illustrates an event (toe off) in which the toe of the right foot is separated from the ground while the sole of the left foot is in contact with the ground (TO: Toe Off). FIG. 4(f) illustrates an event (foot adjacent) in which the left foot and the right foot cross each other while the sole of the left foot is in contact with the ground (FA: Foot Adjacent). FIG. 4(g) illustrates an event (tibia vertical) in which the tibia of the right foot is substantially perpendicular to the ground while the sole of the left foot is in contact with the ground (TV: Tibia Vertical). FIG. 4(h) illustrates an event (heel strike) in which the heel of the right foot is grounded (HS: Heel Strike). FIG. 4(h) is relevant to the end point of the gait cycle starting from FIG. 4(a), and relevant to the start point of the next gait cycle.

The initial stance period T1 is a period from the heel strike HS to the opposite toe off OTO. The mid-stance period T2 is a period from the opposite toe off OTO to the heel rise HR. The terminal stance period T3 is a period from the heel rise HR to the opposite heel strike OHS. The pre-swing period T4 is a period from the opposite heel strike OHS to the toe off TO. The initial swing period T5 is a period from the toe off TO to the foot adjacent FA. The mid-swing period T6 is a period from the foot adjacent FA to the tibia vertical TV. The terminal swing period T7 is a period from the tibia vertical TV to the heel strike HS. The timing at which the gait event appears differs depending on a person, a physical condition, and a gait state, and thus does not completely coincide with the assumed gait cycle.

In the gait motion, a periodic motion is repeated as illustrated in FIG. 4. Therefore, the skeleton and the muscle used at the same timing (also referred to as a gait phase) of different gait cycles are almost the same. Therefore, the waveforms of the time-series data of the sensor data measured along with gait are similar in each gait phase. In the present example embodiment, the missing section of the time-series data of the gait cycle of the missing interpolation target is interpolated using the data without missing included in the time-series data of the gait cycle different from the gait cycle of the missing interpolation target.

The interpolation device 12 receives sensor data from the measurement device 11. The interpolation device 12 generates time-series data of the received sensor data. The interpolation device 12 generates time-series data based on the time stamp included in the sensor data. The interpolation device 12 cuts out a waveform for one gait cycle from the generated time-series data. For example, the interpolation device 12 cuts out a waveform for one gait cycle starting from the timing of the heel strike or the heel rise as a start point. The interpolation device 12 normalizes the cut-out waveform for one gait cycle to generate a waveform (also referred to as a gait waveform) for one gait cycle.

The interpolation device 12 specifies a portion of the time-series data of the sensor data in which the number of the time stamp is missing as a missing section. The interpolation device 12 generates interpolation data for interpolating data of the missing section using a gait waveform in another gait cycle without a missing in the same gait phase as the missing section. The interpolation device 12 interpolates the missing section using the generated interpolation data.

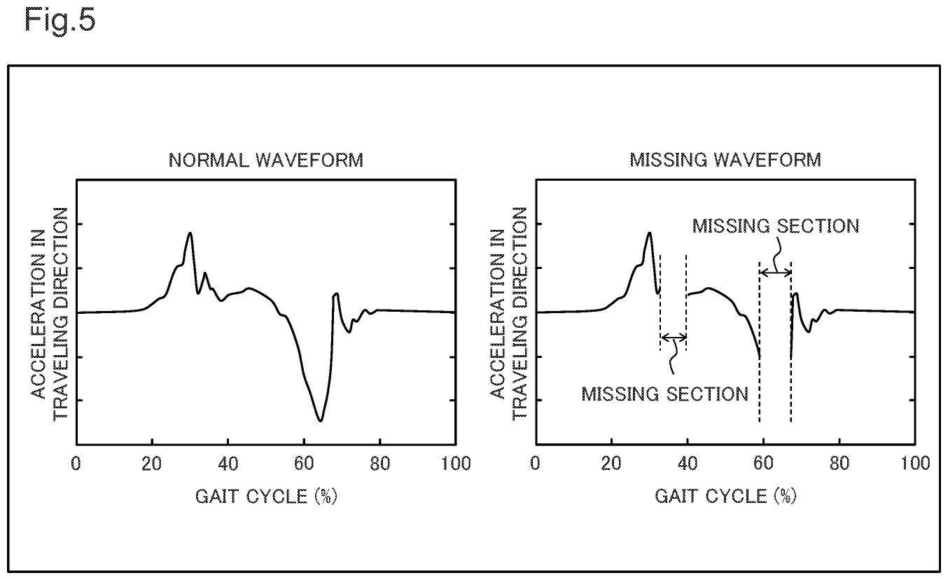

FIG. 5 is a conceptual diagram for comparing a normal waveform with a waveform including a missing (also referred to as a missing waveform). FIG. 5 is a gait waveform of the acceleration in the traveling direction (Y-direction acceleration) in one gait cycle with the heel rise as a start point. The missing waveform in FIG. 5 includes two missing sections. As illustrated in FIG. 5, the data of the missing section may include the feature of gait.

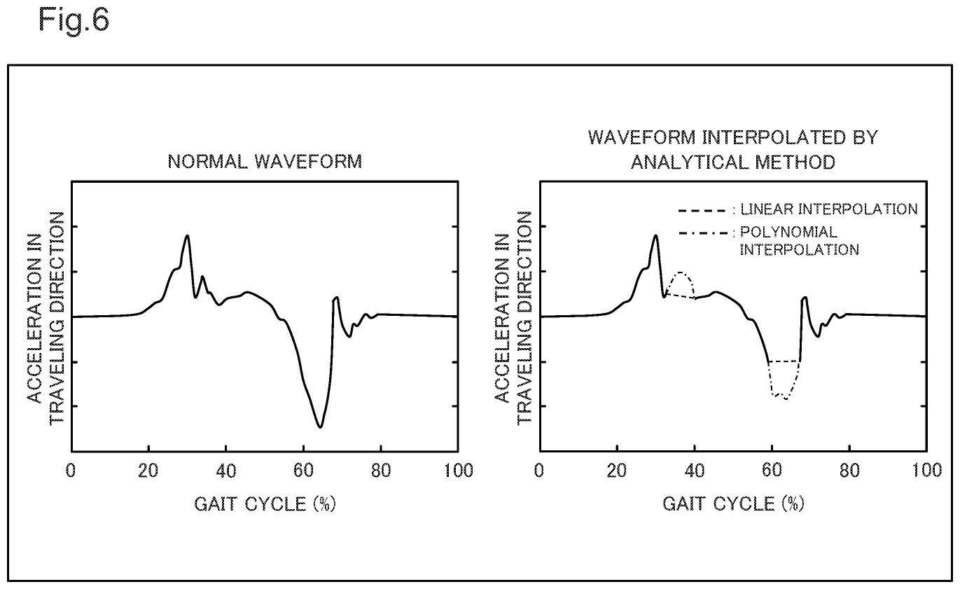

FIG. 6 is a conceptual diagram for explaining an example of interpolation of a missing section of time-series data in the related art. FIG. 6 illustrates an example in which data in a missing section is interpolated by an analytical method with reference to a time stamp of sensor data included in time-series data. FIG. 6 illustrates linear interpolation (broken line) in which the start point and the end point of the missing section are connected by a line segment (linear function) and polynomial interpolation (alternate long and short dash line) in which the start point and the end point of the missing section are connected by a curve. In the linear interpolation, a start point and an end point of a missing section are connected by a line segment. In the linear interpolation, since the unevenness of the amplitude appearing in the time-series data cannot be restored, the feature of the section including the unevenness cannot be restored. In the polynomial interpolation, a start point and an end point of a missing section are connected by a curve. In the polynomial interpolation, although the unevenness of the time-series data can be roughly restored, fine features included in the unevenness portion cannot be restored.

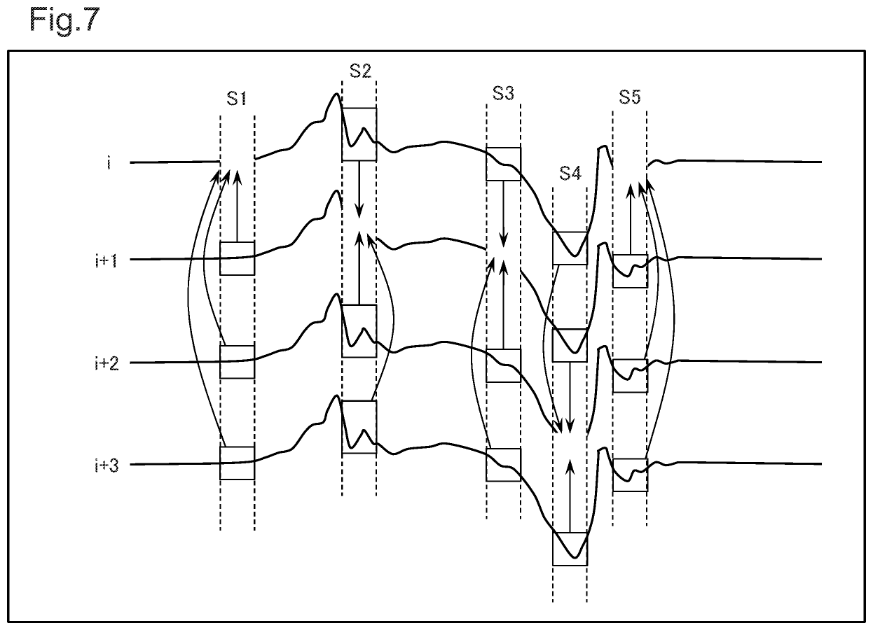

FIG. 7 is a conceptual diagram for explaining an example of restoration of a missing section of time-series data in the present example embodiment. In the example of FIG. 7, the data of missing sections S1 to S5 included in each of the gait waveforms of the gait cycles i to i+3 is interpolated with each other (i is a natural number). The gait waveform in the gait cycle i includes the missing section S1 and the missing section S5. The gait waveform in the gait cycle i+1 includes the missing section S2 and the missing section S3. The gait waveform in the gait cycle i+2 includes the missing section S4. The gait waveform in the gait cycle i+3 does not include the missing section.

The interpolation device 12 interpolates the missing section S1 included in the gait waveform of the gait cycle i by using the data of the gait phase relevant to the missing section S1 included in the gait waveform of the gait cycles i+1 to i+3. The interpolation device 12 interpolates the missing section S5 included in the gait waveform of the gait cycle i by using the data of the gait phase relevant to the missing section S5 included in the gait waveform of the gait cycles i+1 to i+3. The interpolation device 12 interpolates the missing section S2 included in the gait waveform of the gait cycle i+1 with the data of the gait phase relevant to the missing section S2 included in the gait waveform of the gait cycles i, i+2, and i+3. The interpolation device 12 interpolates the missing section S3 included in the gait waveform of the gait cycle i+1 with the data of the gait phase relevant to the missing section S3 included in the gait waveform of the gait cycles i, i+2, and i+3. The interpolation device 12 interpolates the missing section S4 included in the gait waveform of the gait cycle i+2 with the data of the gait phase relevant to the missing section S4 included in the gait waveforms of the gait cycles i, i+1, and i+3.

For example, regarding a missing section of time-series data generated in certain time-series data, the interpolation device 12 selects at least one piece of data of a gait phase relevant to the missing section from at least one gait waveform in which the data of the missing section is not missing. For example, the interpolation device 12 generates interpolation data for interpolating the missing section using the selected data.

For example, regarding a missing section of time-series data generated in certain time-series data, the interpolation device 12 selects a plurality of pieces of data of the gait phase relevant to the missing section from a plurality of gait waveforms in which the data of the missing section is not missing. The interpolation device 12 calculates an average value such as an addition average or a weighted average for the plural pieces of selected data, and generates interpolation data for interpolating the missing section using the calculated average value. For example, the interpolation device 12 generates interpolation data by using data having the closest values at the start point and the end point of the missing section among the plural pieces of selected data.

For example, the interpolation device 12 may select a method of interpolating the data of the missing section according to the gait period of the missing section. For example, the waveform of the acceleration (Y-direction acceleration) of progressive walking has a small fluctuation during the period of the stance phase and a large fluctuation during the period of the swing phase. Therefore, the interpolation device 12 may be configured to interpolate the data of the missing section by an analytical method for the missing section generated in the stance phase, and interpolate the data of the missing section based on the gait waveform of another gait cycle for the missing section generated in the swing phase.

For example, the interpolation device 12 is mounted on a server (not illustrated) or the like. For example, the interpolation device 12 may be achieved by an application server. For example, the interpolation device 12 may be achieved by application software or the like installed in a mobile terminal (not illustrated).

The gait measurement device 13 acquires a gait waveform not including the missing section from the interpolation device 12. The gait measurement device 13 executes measurement regarding the gait using the acquired gait waveform. For example, the gait measurement device 13 detects a gait event from the gait waveform, and measures the gait of the user based on the detected gait event. For example, the gait measurement device 13 measures a gait such as a stride length, a step length, a stride, a step width, a foot angle, and a gait speed based on the detected gait event. For example, the gait measurement device 13 may estimate a physical state such as a degree of pronation/supination, a degree of progress of hallux valgus, body symmetry, and body flexibility based on the measured gait. For example, the gait measurement device 13 may measure a body parameter such as a length of an upper limb, a lower limb, an upper arm, a forearm, a thigh, or a lower leg based on the measured gait. The measurement target item by the gait measurement device 13 is not particularly limited.

The gait measurement device 13 outputs a measurement result (also referred to as gait information) regarding the gait. For example, the gait measurement device 13 outputs the gait information to a display device (not illustrated) or a mobile terminal (not illustrated). The gait information output to the display device is displayed on a screen of the display device or the mobile terminal. For example, the gait measurement device 13 outputs the gait information to an external system (not illustrated). The gait information output from the gait measurement device 13 can be used for any purpose. The communication function of outputting the gait information by the gait measurement device 13 is not particularly limited.

For example, the gait measurement device 13 is mounted on a server (not illustrated) or the like. For example, the gait measurement device 13 may be achieved by an application server. For example, the gait measurement device 13 may be achieved by application software or the like installed in a mobile terminal (not illustrated).

### [Measurement Device]

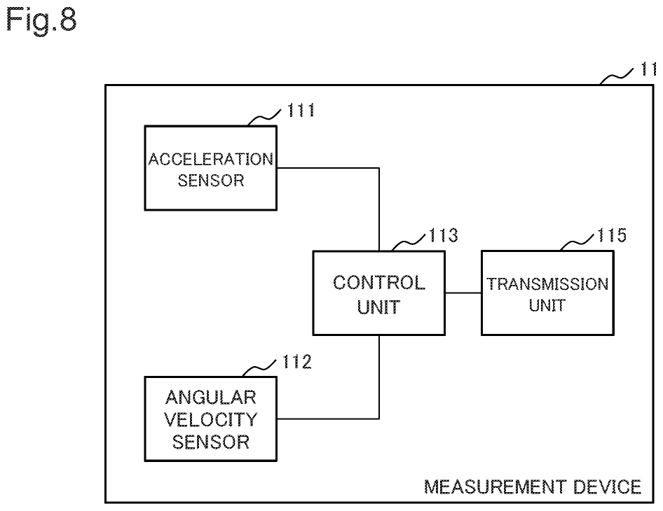

Next, the detailed configuration of the measurement device 11 will be described with reference to the drawings. FIG. 8 is a block diagram illustrating an example of the detailed configuration of the measurement device 11. The measurement device 11 includes an acceleration sensor 111, an angular velocity sensor 112, a control unit 113, and a transmission unit 115. The measurement device 11 includes a power supply (not illustrated).

The acceleration sensor 111 is a sensor that measures accelerations (also referred to as spatial accelerations) in three axial directions. The acceleration sensor 111 outputs the measured acceleration to the control unit 113. For example, a sensor of a piezoelectric type, a piezoresistive type, a capacitance type, or the like can be used as the acceleration sensor 111. The sensor used for the acceleration sensor 111 is not limited to the measurement method as long as the sensor can measure acceleration.

The angular velocity sensor 112 is a sensor that measures an angular velocity (also referred to as a spatial angular velocity) in the three axial directions. The angular velocity sensor 112 outputs the measured angular velocity to the control unit 113. For example, a sensor of a vibration type, a capacitance type, or the like can be used as the angular velocity sensor 112. The sensor used for the angular velocity sensor 112 is not limited to the measurement method as long as the sensor can measure the angular velocity.

The control unit 113 acquires the acceleration in the three axial directions and the angular velocity around three axes from each of the acceleration sensor 111 and the angular velocity sensor 112. The control unit 113 converts the acquired acceleration and angular velocity into digital data, and outputs the converted digital data (also referred to as sensor data) to the transmission unit 115. The sensor data includes at least acceleration data converted into digital data and angular velocity data converted into digital data. The acceleration data includes acceleration vectors in three axial directions. The angular velocity data includes angular velocity vectors around three axes. The acceleration data and the angular velocity data are associated with acquisition times of the data. The control unit 113 may be configured to output sensor data obtained by adding correction such as a mounting error, temperature correction, and linearity correction to the acquired acceleration data and angular velocity data. The control unit 113 may generate angle data around three axes using the acquired acceleration data and angular velocity data.

For example, the control unit 113 is a microcomputer or a microcontroller that controls the overall measurement device 11 or processes data. For example, the control unit 113 includes a central processing unit (CPU), a random access memory (RAM), a read only memory (ROM), a flash memory, and the like. The control unit 113 controls the acceleration sensor 111 and the angular velocity sensor 112 to measure the angular velocity and the acceleration. For example, the control unit 113 performs analog-to-digital conversion (AD conversion) on the acquired physical quantities (analog data) such as measured angular velocity and acceleration. The control unit 113 stores the converted digital data in a storage unit (not illustrated) such as a flash memory. The physical quantity (analog data) measured by the acceleration sensor 111 and the angular velocity sensor 112 may be converted into digital data in each of the acceleration sensor 111 and the angular velocity sensor 112. The digital data stored in the storage unit is output to the transmission unit 115 at a predetermined timing.

The transmission unit 115 acquires sensor data from the control unit 113. The transmission unit 115 transmits the acquired sensor data to the interpolation device 12. For example, the transmission unit 115 transmits the sensor data to the interpolation device 12 via a wire such as a cable. For example, the transmission unit 115 transmits the sensor data to the interpolation device 12 via wireless communication. For example, the transmission unit 115 is configured to transmit the sensor data to the interpolation device 12 via a wireless communication function (not illustrated) conforming to a standard such as Bluetooth (registered trademark) or WiFi (registered trademark). The communication function of the transmission unit 115 may conform to a standard other than Bluetooth (registered trademark) or WiFi (registered trademark).

### [Interpolation Device]

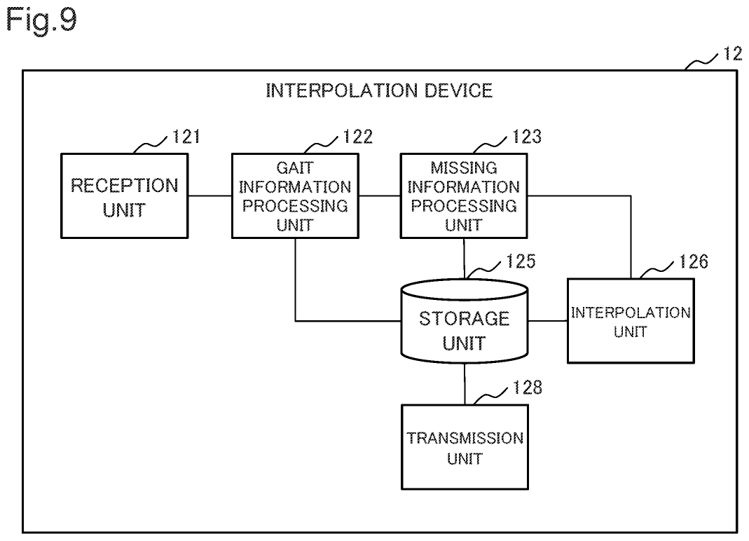

Next, a detailed configuration of the interpolation device 12 will be described with reference to the drawings. FIG. 9 is a block diagram for explaining the detailed configuration of the interpolation device 12. The interpolation device 12 includes a reception unit 121, a gait information processing unit 122, a missing information processing unit 123, a storage unit 125, an interpolation unit 126, and a transmission unit 128.

The reception unit 121 receives sensor data from the measurement device 11. The reception unit 121 outputs the received sensor data to the gait information processing unit 122. For example, the reception unit 121 receives the sensor data from the measurement device 11 via a wire such as a cable. For example, the reception unit 121 receives sensor data from the measurement device 11 via wireless communication. For example, the reception unit 121 is configured to receive the sensor data to the measurement device 11 via a wireless communication function (not illustrated) conforming to a standard such as Bluetooth (registered trademark) or WiFi (registered trademark). The communication function of the reception unit 121 may conform to a standard other than Bluetooth (registered trademark) or WiFi (registered trademark).

The gait information processing unit 122 acquires, from the reception unit 121, the sensor data measured along with gait of the user wearing the shoe 100 on which the measurement device 11 is installed. The gait information processing unit 122 generates time-series data of the acquired sensor data based on the time stamp included in the sensor data. For example, the gait information processing unit 122 converts the coordinate system of the acquired sensor data from the local coordinate system to the world coordinate system.

The gait information processing unit 122 generates time-series data of a physical quantity regarding the motion of the foot using the sensor data. For example, the gait information processing unit 122 generates time-series data such as a spatial acceleration and a spatial angular velocity. The gait information processing unit 122 integrates the spatial acceleration and the spatial angular velocity to generate time-series data such as a spatial speed, a spatial angle (plantar angle), and a spatial trajectory. These time-series data correspond to the gait waveform. The gait information processing unit 122 generates time-series data at a predetermined timing or time interval set in accordance with a general gait cycle or a gait cycle unique to the user. The timing at which the gait information processing unit 122 generates the time-series data can be arbitrarily set. For example, the gait information processing unit 122 is configured to continue to generate time-series data during a period in which gait of the user is continued. The gait information processing unit 122 may be configured to generate time-series data at a specific timing.

The gait information processing unit 122 cuts out a waveform corresponding to one gait cycle from the generated time-series data. For example, the gait information processing unit 122 cuts out a waveform corresponding to one gait cycle with the heel strike as a start point. For example, the gait information processing unit 122 detects the timing of the heel strike in the gait waveform of the acceleration in the traveling direction (Y-direction acceleration) of one gait cycle. In the gait waveform of the acceleration in the traveling direction for one gait cycle, the timing of the heel strike is the timing of the midpoint between the timing at which the minimum peak is detected and the timing at which the maximum peak appearing after the minimum peak is detected. For example, the gait information processing unit 122 cuts out time-series data with the preceding heel strike as a start point and the subsequent heel strike as an end point as a waveform for one gait cycle with the heel strike as a start point.

For example, the gait information processing unit 122 detects the timing of the toe off in the gait waveform of the acceleration in the traveling direction for one gait cycle. In the gait waveform of the acceleration in the traveling direction for one gait cycle, the timing of the toe off is the timing of the valley appearing between the two mountains included in the maximum peak. For example, the gait information processing unit 122 detects the timing of the valley appearing between two mountains included in the maximum peak in the gait waveform of the acceleration in the traveling direction for one gait cycle as the timing of the toe off.

For example, the gait information processing unit 122 may extract a waveform for one gait cycle with the heel rise as a start point. In the gait waveform of the plantar angle, a state in which the toe is located above the heel (dorsiflexion) is defined as negative, and a state in which the toe is located below the heel (plantarflexion) is defined as positive. The time at which the gait waveform of the plantar angle becomes minimum corresponds to the timing of the start of the stance phase. The time at which the gait waveform of the plantar angle becomes maximum corresponds to the timing of the start of the swing phase. The time at the midpoint between the time of the start of the stance phase and the time of the start of the swing phase corresponds to the center timing of the stance phase. The timing at the center of the stance phase corresponds to the timing of the heel rise. For example, the gait information processing unit 122 cuts out time-series data having a preceding timing as a start point and a subsequent timing as an end point among two heel rise timings included in the time-series data for two walks. The cut-out time-series data is a gait waveform for one gait cycle with the heel rise as a start point.

The gait information processing unit 122 normalizes the cut waveform for one gait cycle and generates a waveform (also referred to as a gait waveform) for one gait cycle. The gait information processing unit 122 normalizes the time axis of the waveform corresponding to one gait cycle to a gait cycle in which the timing of the start point is 0 and the timing of the end point is 100. The gait cycle of the normalized waveform is divided into 100 equal parts and expressed as a percentage. Each percentage of the gait cycle is referred to as a gait phase. For example, the gait information processing unit 122 normalizes the gait cycle of the gait waveform such that the ratio between the stance phase and the swing phase is 60:40. For example, the gait information processing unit 122 may normalize the gait waveform based on the gait event appearing in the gait waveform. For example, with respect to the gait waveform with the timing of the heel rise as a start point, the gait information processing unit 122 normalizes the gait waveform such that the timing of the toe off becomes 30% and the timing of the heel strike becomes 70%. The timing at which the gait event appears differs depending on the person, the physical condition, and the gait state, and thus does not completely coincide with the assumed gait phase.

The gait information processing unit 122 may normalize the amplitude (value of acceleration or angular velocity) of the gait waveform for one gait cycle. For example, with respect to a gait waveform for one gait cycle, the gait information processing unit 122 normalizes the amplitude of the gait waveform so that a fluctuation range of the amplitude falls within a certain range. For example, the gait information processing unit 122 normalizes the amplitude of the gait waveform so that the maximum value of the amplitude becomes 1 and the maximum value of the amplitude becomes −1 with respect to the gait waveform for one gait cycle. If the amplitude of the gait waveform is normalized, the amplitudes of the gait waveforms of a plurality of gait cycles are aligned, so that it is easy to interpolate data to the missing section. The gait information processing unit 122 may normalize the baseline of the gait waveform for one gait cycle. For example, the gait information processing unit 122 normalizes the gait waveform for one gait cycle so that the inclination of the baseline becomes 0. When the baseline of the gait waveform is normalized, the inclinations of the baselines of the gait waveforms in a plurality of gait cycles are aligned. Therefore, if the baseline of the gait waveform is normalized, it is easy to interpolate the data of the missing section.

The gait information processing unit 122 stores the normalized gait waveform in the storage unit 125. For example, the gait information processing unit 122 stores only the gait waveform without the missing section in the storage unit 125. In this case, a gait waveform having a missing section (also referred to as a missing waveform) may be stored in the storage unit 125 after the missing information processing unit 123 specifies the missing section. For example, all the gait waveforms including the missing waveform may be stored in the storage unit 125. In that case, the missing information processing unit 123 may acquire the missing waveform stored in the storage unit 125 and specify the missing section included in the missing waveform. The data (also referred to as normal data) of the section without the missing of the gait waveform stored in the storage unit 125 is used to interpolate the missing section included in the missing waveform.

The missing information processing unit 123 acquires a missing waveform. The missing information processing unit 123 may acquire the missing waveform from the gait information processing unit 122 or may acquire the missing waveform from the storage unit 125. The missing information processing unit 123 specifies a portion in which the number of the time stamp is missing in the acquired missing waveform as a missing section. For example, the missing information processing unit 123 determines which data is missing from the start point of the missing section in the gait waveform of one gait cycle, and calculates the missing gait phase. For example, the missing information processing unit 123 may specify the gait period of the missing section based on the number of the time stamp.

The missing information processing unit 123 stores information regarding the missing section included in the missing waveform (also referred to as missing information) in the storage unit 125 in association with the missing waveform. For example, the missing information processing unit 123 stores, as the missing information, the gait phases at the start point and the end point of the missing section in the storage unit 125 in association with the missing waveform. For example, the missing information processing unit 123 may store, as the missing information, the gait phase at the start point and the end point of the missing section and the value of the amplitude in the gait phase in the storage unit 125 in association with the missing waveform. The missing information processing unit 123 may be configured to output missing information to the interpolation unit 126.

The gait waveform is stored in the storage unit 125. The storage unit 125 stores a gait waveform (also referred to as a normal waveform) not including the missing section. A gait waveform including the missing section (also referred to as a missing waveform) and a gait waveform in which the missing section is repaired (also referred to as a restoration waveform) are stored in the storage unit 125. The missing waveform is an interpolation target of the missing section. The missing waveform is associated with missing information of the missing waveform. The normal waveform and the restoration waveform are used for gait measurement by the gait measurement device 13. The gait waveform stored in the storage unit 125 is acquired by the transmission unit 128 at a predetermined timing and transmitted from the transmission unit 128 to the gait measurement device 13.

The interpolation unit 126 acquires a missing waveform. The interpolation unit 126 acquires the missing waveform from the storage unit 125 or the missing information processing unit 123. The missing waveform acquired by the interpolation unit 126 includes information (missing information) related to the missing section specified by the missing information processing unit 123. The interpolation unit 126 interpolates the missing section included in the missing waveform using the data of the gait waveform in the gait cycle different from the acquired missing waveform. The interpolation unit 126 extracts the normal data in the gait phase relevant to the missing section from the gait waveform having the gait cycle different from the missing waveform. The interpolation unit 126 generates interpolation data for interpolating the missing section using the extracted interpolation data.

For example, it is assumed that the missing waveform includes the missing section in the section in which the gait phase is 10 to 20%. In this case, the interpolation unit 126 selects at least one gait waveform that does not include a defect in a section in which the gait phase is 10 to 20% among the gait waveforms stored in the storage unit 125. The interpolation unit 126 extracts normal data in a section in which the gait phase is 10 to 20% from the selected at least one gait waveform. For example, with respect to a missing section included in a certain missing waveform, the interpolation unit 126 selects at least one normal data of the gait phase relevant to the missing section from at least one gait waveform in which the data of the missing section is not missing. The interpolation unit 126 generates interpolation data for interpolating the missing section using the selected normal data.

For example, with respect to a missing section included in a certain missing waveform, the interpolation unit 126 selects a plurality of pieces of data of the gait phase relevant to the missing section from a plurality of gait waveforms in which data of the missing section is not missing. For example, the interpolation unit 126 calculates interpolation data for each single gait phase included in the missing section. The interpolation unit 126 calculates an average value such as an addition average or a weighted average for the plural pieces of selected data, and generates interpolation data for interpolating the missing section using the calculated average value. For example, the interpolation unit 126 generates interpolation data by using data having the closest values at the start point and the end point of the missing section among the plural pieces of selected data.

For example, the interpolation unit 126 may select a method of interpolating the data of the missing section according to the gait period of the missing section. For example, the waveform of the acceleration (Y-direction acceleration) of progressive walking has a small fluctuation during the period of the stance phase and a large fluctuation during the period of the swing phase. That is, in the period of the swing phase (also referred to as a first gait period), data variation is greater than in the period of the stance phase (also referred to as a second gait period), and thus it is difficult to analytically interpolate data. In other words, in the period of the stance phase (second gait period), data variation is smaller than that in the period of the swing phase (first gait period), and thus data can be interpolated analytically. Therefore, the interpolation unit 126 may interpolate the missing section included in the period of the stance phase (second gait period) by an analytical method. The missing section included in the period of the swing phase (first gait period) may be interpolated based on the gait waveform of another gait cycle. The interpolation unit 126 may also interpolate a missing section included in the swing phase (first gait period) by an analytical method as long as the section has a small fluctuation.

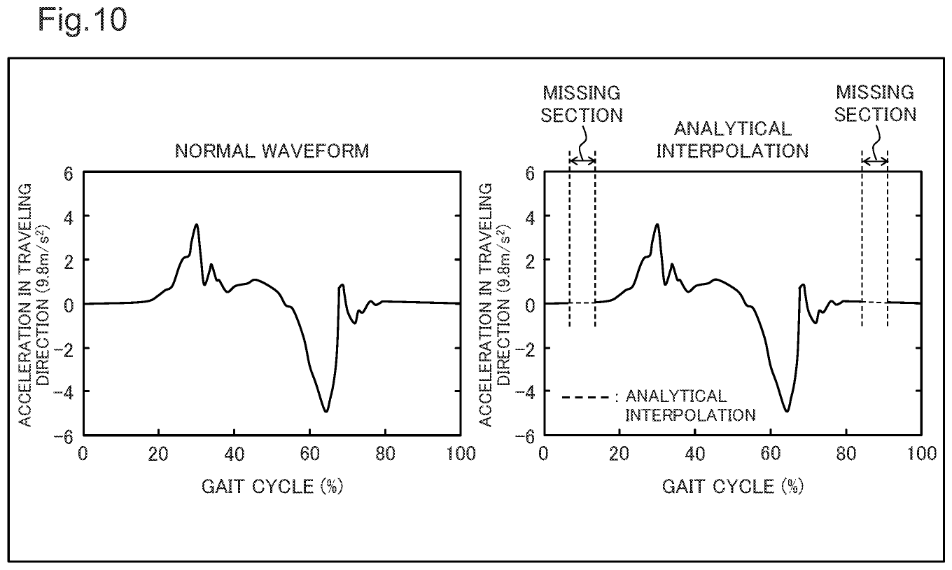

FIG. 10 is a conceptual diagram for explaining interpolation of missing sections in analytically interpolated sections. In the example of FIG. 10, the fluctuation of the amplitude is small in a section in which the gait phase is 0 to 20% or a section in which the gait phase is 80% to 100%. On the other hand, the fluctuation of the amplitude is large in a section in which the gait phase is 20 to 80%. In such a case, the interpolation unit 126 may perform analytical interpolation for a section in which the gait phase is 0 to 20% or a section in which the gait phase is 80% to 100%. For a section in which the gait phase is 20 to 80%, since the fluctuation in the amplitude is large, the interpolation unit 126 may generate interpolation data using data of other gait waveforms.

The interpolation unit 126 interpolates the missing section included in the missing waveform using the generated interpolation data. For example, the interpolation unit 126 interpolates the missing waveform by inserting the interpolation data into the missing section. For example, the interpolation unit 126 sequentially interpolates the missing section with the normal data included in the interpolation data from the start point to the end point of the missing section. For example, the interpolation unit 126 may sequentially interpolate the missing section with the normal data included in the interpolation data from the end point to the start point of the missing section. For example, the interpolation unit 126 may sequentially interpolate the normal data included in the interpolation data from the end point and the start point of the missing section.

For example, the interpolation unit 126 inserts a waveform configured by interpolation data into the missing section. For example, the interpolation unit 126 inserts a waveform configured by the interpolation data such that the start point and the end point of the interpolation data coincide with the start point and the end point of the missing section, respectively. For example, it is assumed that the start point and the end point of the interpolation data do not coincide with the start point and the end point of the missing section. In such a case, the interpolation unit 126 rotates the baseline of the interpolation data, and inserts the interpolation data into the missing section so that the start point and the end point of the interpolation data coincide with the start point and the end point of the missing section. For example, the interpolation unit 126 calculates the amplitudes of the start point and the end point of the missing section based on the normal waveforms of other gait cycles. For example, the interpolation unit 126 adjusts the baseline according to a difference between the calculated amplitudes of the start point and the end point of the missing section and the actual amplitudes of the start point and the end point of the missing section.

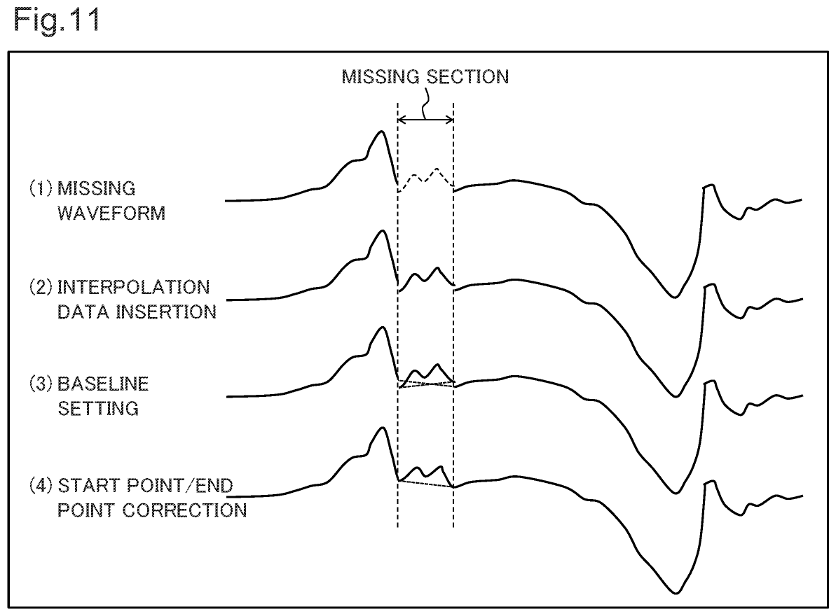

FIG. 11 is a conceptual diagram illustrating an example in which a waveform configured by interpolation data is inserted into a missing section. (1) The interpolation unit 126 generates interpolation data of the missing section included in the missing waveform. (2) The interpolation unit 126 inserts the generated interpolation data into the missing section of the missing waveform. (3) The interpolation unit 126 sets a baseline in the interpolation data inserted into the missing section of the missing waveform. (4) The interpolation unit 126 rotates the baseline of the interpolation data inserted into the missing section of the missing waveform, and performs correction of the start point and the end point such that the start point and the end point of the interpolation data coincide with the start point and the end point of the missing section, respectively.

The interpolation unit 126 stores the gait waveform (restoration waveform) in which the missing section is interpolated in the storage unit 125. For example, the interpolation unit 126 may attach a label to the gait phase included in the missing section so that the repaired missing section can be specified in the restoration waveform. For example, the interpolation unit 126 may cause the storage unit 125 to store a restoration waveform (also referred to as a temporary restoration waveform) in which temporary interpolation data is inserted into the missing section. For example, the gait measurement device 13 may correct the interpolation data inserted into the missing section included in the temporary restoration waveform. The restoration waveform stored in the storage unit 125 is used for the gait measurement by the gait measurement device 13, similarly to other gait waveforms not including a defect.

The transmission unit 128 acquires the gait waveform from the storage unit 125. The transmission unit 128 acquires the gait waveform including the normal waveform and the restoration waveform from the storage unit 125. The transmission unit 128 transmits the acquired gait waveform to the gait measurement device 13. For example, the transmission unit 128 transmits the gait waveform to the gait measurement device 13 via a wire such as a cable. For example, the transmission unit 128 transmits the gait waveform to the gait measurement device 13 via wireless communication. A communication system between the transmission unit 128 and the gait measurement device 13 is not particularly limited.

### [Gait Measurement Device]

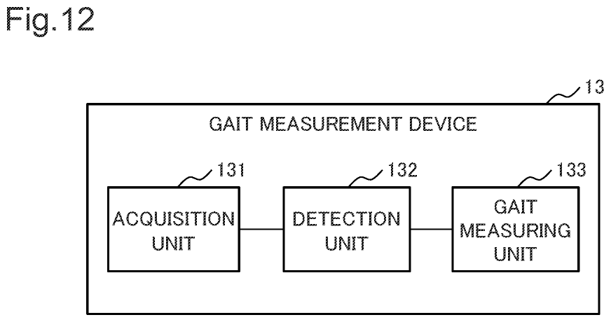

Next, a detailed configuration of the gait measurement device 13 included in the gait measurement system 1 will be described with reference to the drawings. FIG. 12 is a block diagram illustrating an example of the detailed configuration of the gait measurement device 13. The gait measurement device 13 includes an acquisition unit 131, a detection unit 132, and a gait measuring unit 133. Actually, an output unit (communication interface) that outputs an estimation result by the gait measuring unit 133 is provided, but in FIG. 12, the function of the output unit is included in the gait measuring unit 133.

The acquisition unit 131 acquires a gait waveform from the interpolation device 12. The acquisition unit 131 acquires the gait waveform including the normal waveform and the restoration waveform from the interpolation device 12. The acquisition unit 131 outputs the acquired gait waveform to the detection unit 132. For example, the acquisition unit 131 receives the gait waveform from the interpolation device 12 via a wire such as a cable. For example, the acquisition unit 131 receives the gait waveform from the interpolation device 12 via wireless communication. A communication system between the interpolation device 12 and the acquisition unit 131 is not particularly limited.

The detection unit 132 acquires the gait waveform from the acquisition unit 131. The detection unit 132 detects a gait period and a gait event from the acquired gait waveform. For example, the detection unit 132 detects a gait period such as a stance phase and a swing phase from the gait waveform. For example, the detection unit 132 detects a gait event such as a heel strike, an opposite toe off, a heel rise, an opposite heel strike, a toe off, a foot adjacent, and a tibia vertical from the gait waveform. For example, based on the detected gait event, the detection unit 132 detects a gait period such as an initial stance period, a mid-stance period, a terminal stance period, a pre-swing period, an initial swing period, a mid-swing period, and a terminal swing period from the gait waveform.

For example, the detection unit 132 detects a gait period or a gait event from the gait waveform based on the percentage of the gait phase. If the same person is walking under the same condition, the timing at which the gait period or the gait event is detected is substantially constant. Even if the person is different, the gait period and the timing at which the gait event is detected show the same tendency. Therefore, the gait period and the gait event can be specified based on the percentage of the gait phase.

For example, the detection unit 132 may detect a gait period or a gait event based on a feature extracted from the gait waveform. For example, the detection unit 132 detects the timing at which the gait waveform of the plantar angle becomes minimum as the timing of the start of the stance phase. For example, the detection unit 132 detects the timing at which the gait waveform of the plantar angle becomes maximum as the timing of the start of the swing phase. For example, the detection unit 132 detects the timing of the midpoint between the start of the stance phase and the start of the swing phase as the timing of the heel rise.

For example, the detection unit 132 detects the gait event based on the gait waveform with the timing of the heel rise as a start point. The detection unit 132 detects the timing at which a valley appears between two mountains included in the maximum peak in the gait waveform of the acceleration in the traveling direction (Y-direction acceleration) as the timing of the toe off. For example, the detection unit 132 detects a minimum peak around when the gait cycle exceeds 60% in the gait waveform of the acceleration in the traveling direction (Y-direction acceleration) as the timing of sudden deceleration of the foot at the terminal swing period. For example, the detection unit 132 detects a maximum peak around a gait cycle of 70% in the gait waveform of the acceleration in the traveling direction (Y-direction acceleration) as the timing of a heel rocker. For example, the detection unit 132 detects the timing of the midpoint between the minimum peak and the maximum peak in the gait waveform of the acceleration in the traveling direction (Y-direction acceleration) as the timing of heel strike. For example, in the gait waveform of the acceleration in the vertical direction (Z-direction acceleration), the detection unit 132 detects the timing of the maximum peak between the toe off and the heel strike as the timing of the tibia vertical. For example, the detection unit 132 detects the timing of a gentle maximum peak between the timing of the toe off and the timing of the tibia vertical in the gait waveform of the acceleration in the traveling direction (Y-direction acceleration) as the timing of the foot adjacent. For example, the detection unit 132 detects the timing of the opposite heel strike and the opposite toe off from the gait waveform of the roll angular velocity. For example, in the gait waveform of the roll angular velocity, the detection unit 132 detects the timing of the acceleration inflection point in the curve from the start point to the timing of the toe off as the timing of the opposite heel strike. For example, in the gait waveform of the roll angular velocity, the detection unit 132 detects the timing of the deceleration inflection point in the curve from the timing of heel strike to the end point as the timing of the opposite toe off.

The gait measuring unit 133 acquires information regarding the gait period and the gait event detected by the detection unit 132. The gait measuring unit 133 measures the gait using the information acquired from the detection unit 132. For example, the gait measuring unit 133 measures the gait of the user based on the gait event detected from the gait waveform. For example, the gait measuring unit 133 measures a gait such as a stride length, a step length, a stride, a step width, a foot angle, and a gait speed based on the detected gait event. For example, the gait measuring unit 133 may estimate a physical state such as a degree of pronation/supination, a degree of progress of hallux valgus, body symmetry, and body flexibility based on the measured gait. For example, the gait measuring unit 133 may estimate a physical state such as a muscle weakness situation, bone density, and basal metabolism based on the measured gait. For example, the gait measuring unit 133 may measure a body parameter such as lengths of an upper limb, a lower limb, an upper arm, a forearm, a thigh, or a lower leg based on the measured gait. The item to be measured by the gait measuring unit 133 is not particularly limited.

The gait measurement device 13 outputs a result (also referred to as gait information) measured by the gait measuring unit 133. For example, the gait measurement device 13 outputs the gait information to a display device (not illustrated) or a mobile terminal (not illustrated). The gait information output to the display device is displayed on a screen of the display device or the mobile terminal. For example, the gait measurement device 13 outputs the gait information to an external system (not illustrated). The gait information output from the gait measurement device 13 can be used for any purpose. The communication function of outputting the gait information by the gait measurement device 13 is not particularly limited.

### (Operation)

Next, an operation of the interpolation device 12 of the gait measurement system 1 will be described with reference to the drawings. Description of operations of the measurement device 11 and the gait measurement device 13 will be omitted. Hereinafter, processing performed by the gait information processing unit 122, the missing information processing unit 123, and the interpolation unit 126 included in the interpolation device 12 will be individually described. The following operation of the interpolation device 12 may include processing and order different from those of the above-described interpolation device 12.

### [Gait Information Processing]

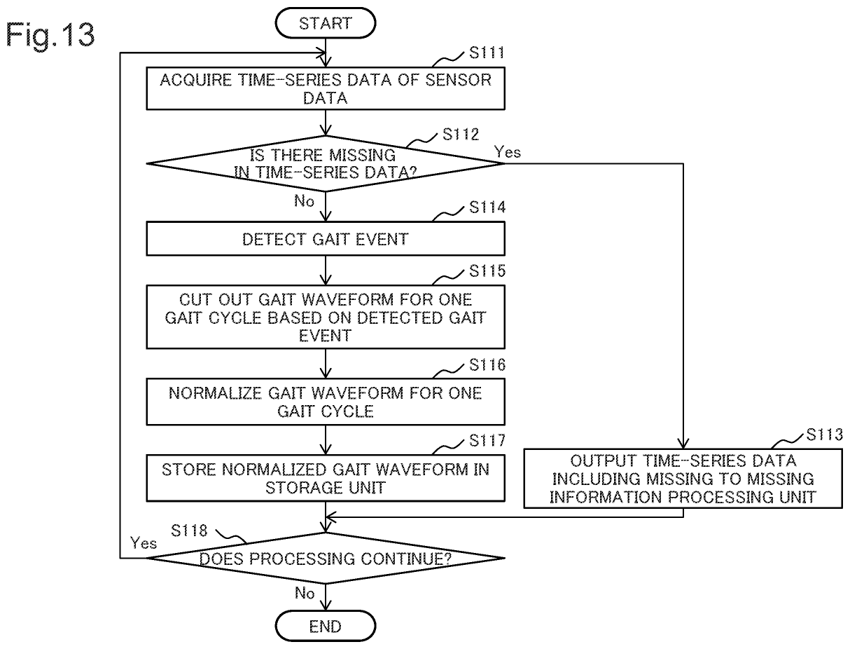

First, the gait information processing by the gait information processing unit 122 will be described with reference to a flowchart. FIG. 13 is a flowchart for explaining the gait information processing by the gait information processing unit 122. In the processing description along the flowchart of FIG. 13, the gait information processing unit 122 will be described as an operation subject.

In FIG. 13, first, the gait information processing unit 122 acquires time-series data of sensor data (step S111).

In a case where there is a missing in the time-series data (Yes in step S112), the gait information processing unit 122 outputs the time-series data including the missing to the missing information processing unit 123 (step S113). The gait information processing unit 122 may store the time-series data including the missing in the storage unit 125. After step S1130, the process proceeds to step S118.

On the other hand, in a case where there is no missing in the time-series data (No in step S112), the gait information processing unit 122 detects a gait event from the acquired time-series data (step S114). For example, the gait information processing unit 122 detects a gait event such as heel strike, toe off, and heel rise from the gait waveform.

After step S114, the gait information processing unit 122 cuts out a gait waveform for one gait cycle based on the detected gait event (step S115). For example, the gait information processing unit 122 cuts out a gait waveform for one gait cycle with the heel strike as a start point. For example, the gait information processing unit 122 cuts out a gait waveform for one gait cycle with the toe off as a start point.

Next, the gait information processing unit 122 normalizes the cut-out gait waveform for one cycle (step S116). For example, the gait information processing unit 122 normalizes the gait phase and amplitude of the gait waveform.

Next, the gait information processing unit 122 stores the normalized gait waveform in the storage unit 125 (step S117). The gait waveform stored in the storage unit 125 is used for interpolation of the missing section included in the missing waveform and gait measurement by the gait measurement device 13.

When the process is continued (Yes in step S118), the process returns to step S111. When the process is not continued (No in step S118), the process according to the flowchart of FIG. 13 is ended. A condition for determining continuation of the process of step S118 may be set in advance.

### [Missing Information Processing]

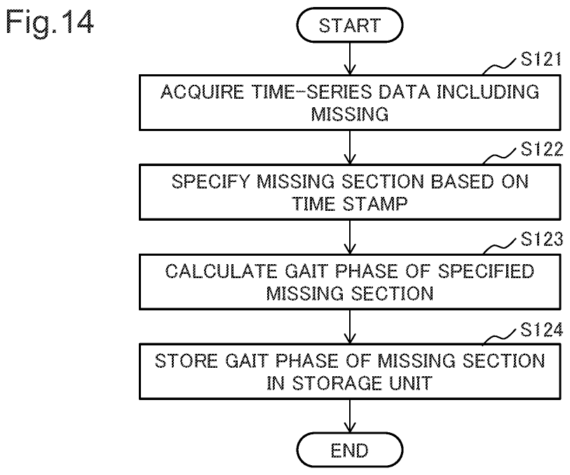

Next, the missing information processing by the missing information processing unit 123 will be described with reference to a flowchart. FIG. 14 is a flowchart for explaining the missing information processing by the missing information processing unit 123. In the processing description along the flowchart of FIG. 14, the missing information processing unit 123 will be described as an operation subject.

In FIG. 14, first, the missing information processing unit 123 acquires time-series data (missing waveform) including a missing from the gait information processing unit 122 (step S121). The missing information processing unit 123 may acquire the missing waveform from the storage unit 125.

Next, the missing information processing unit 123 specifies a missing section included in the acquired missing waveform based on the time stamp (step S122).

Next, the missing information processing unit 123 calculates a gait phase of the specified missing section (step S123).

Next, the missing information processing unit 123 stores information (missing information) regarding the gait phase of the missing section included in the missing waveform in the storage unit 125 (step S124). The missing information stored in the storage unit 125 is used for interpolation of the missing section by the interpolation unit 126.

### [Interpolation Processing]

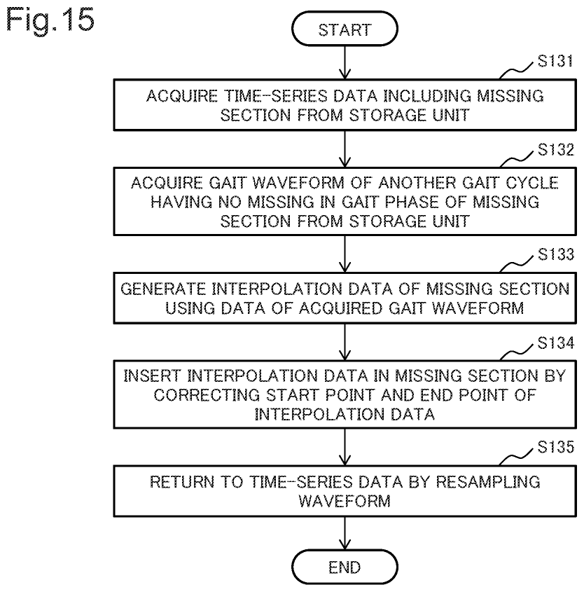

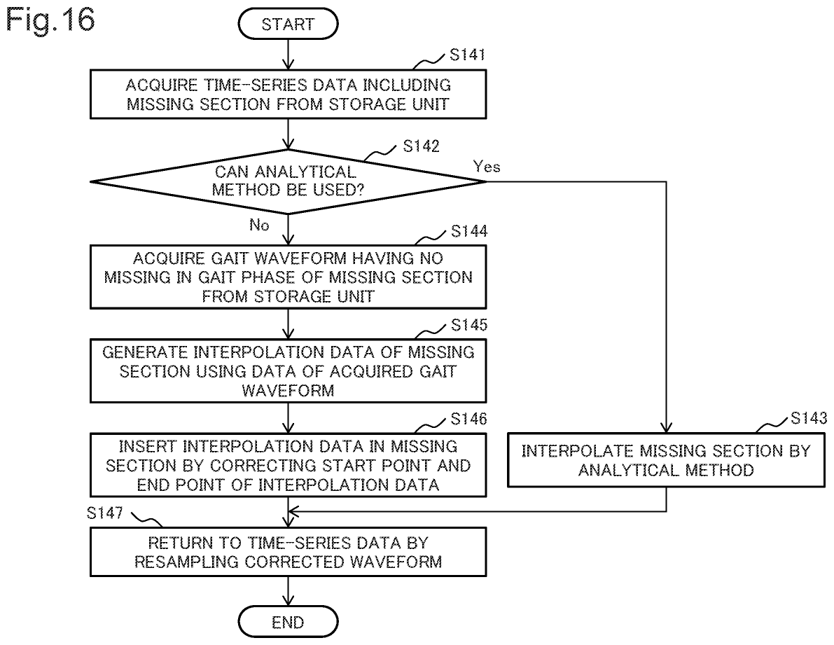

Next, the interpolation processing by the interpolation unit 126 will be described with reference to a flowchart. Here, an example in which interpolation data is generated in all sections (FIG. 15) and an example in which interpolation is performed using an analytical method in some sections (FIG. 16) will be described.

FIG. 15 is a flowchart for explaining the interpolation processing of the interpolation unit 126 in a case where interpolation data is generated in all sections. In the description along the flowchart of FIG. 15, the interpolation unit 126 will be described as an operation subject.

In FIG. 15, first, the interpolation unit 126 acquires time-series data (missing waveform) including the missing section from the storage unit 125 (step S131). The interpolation unit 126 may acquire the missing waveform from the gait information processing unit 122. The missing waveform is associated with missing information indicating the gait phase of the missing section.

Next, the interpolation unit 126 acquires a gait waveform of another gait cycle in which there is no missing in the gait phase of the missing section from the storage unit 125 (step S132).

Next, the interpolation unit 126 generates interpolation data of the missing section using the acquired data of the gait waveform (step S133).

Next, the interpolation unit 126 corrects the start point and the end point of the interpolation data, and inserts the interpolation data into the missing section (step S134).

Next, the interpolation unit 126 resamples the corrected waveform and returns the corrected waveform to the time-series data (step S135). Step S135 is performed because the original time-series data is required when the gait speed, the stride, and the like are calculated. When the original time-series data is not required, step S135 may be omitted.

FIG. 16 is a flowchart for explaining the interpolation processing of the interpolation unit 126 in a case where interpolation is performed using an analytical method in some sections. In the description along the flowchart of FIG. 16, the interpolation unit 126 will be described as an operation subject.

In FIG. 16, first, the interpolation unit 126 acquires time-series data (missing waveform) including the missing section from the storage unit 125 (step S141). The interpolation unit 126 may acquire the missing waveform from the gait information processing unit 122. The missing waveform is associated with missing information indicating the gait phase of the missing section.

In a case where the missing section is a missing section for which an analytical method can be used (Yes in step S142), the interpolation unit 126 interpolates data in the missing section by an analytical method (step S143). After step S143, the process proceeds to step S147.

On the other hand, when the missing section is not a missing section in which the analytical method can be used (No in step S142), the interpolation unit 126 acquires a gait waveform of another gait cycle in which there is no missing in the gait phase of the missing section from the storage unit 125 (step S144).

Next, the interpolation unit 126 generates interpolation data of the missing section using the acquired data of the gait waveform (step S145).

Next, the interpolation unit 126 corrects the start point and the end point of the interpolation data, and inserts the interpolation data into the missing section (step S146).

Next, the interpolation unit 126 resamples the corrected waveform and returns the corrected waveform to the time-series data (step S146). Step S146 is performed because the original time-series data is required when the gait speed, the stride, and the like are calculated. When the original time-series data is not required, step S146 may be omitted.

As described above, the gait measurement system according to the present example embodiment includes the measurement device, the interpolation device, and the gait measurement device. The measurement device is disposed on the user's footwear. The measurement device measures the spatial acceleration and the spatial angular velocity according to the gait of the user. The measurement device generates sensor data based on the measured spatial acceleration and spatial angular velocity. The measurement device outputs the generated sensor data to the interpolation device. The interpolation device includes a reception unit, a gait information processing unit, a missing information processing unit, a storage unit, an interpolation unit, and a transmission unit. The reception unit receives the sensor data transmitted from the measurement device. The gait information processing unit generates time-series data of the sensor data. The gait information processing unit generates a gait waveform for each gait cycle using the time-series data of the sensor data regarding the movement of the foot, and specifies a missing section of the data in the time-series data. The gait information processing unit stores the generated gait waveform and information regarding the specified missing section in the storage unit. The storage unit stores a gait waveform, information regarding a missing section, and a restoration waveform obtained by interpolating the missing section. The missing information processing unit calculates a gait phase of the specified missing section. The interpolation unit generates interpolation data for interpolating the missing section by using the data of the gait phase of the missing section in the gait waveform of the gait cycle different from the gait cycle including the missing section. The interpolation unit interpolates the generated interpolation data in the missing section. The gait measurement device acquires, from the interpolation device, a gait waveform not including the missing section generated by the interpolation device using the time-series data of the sensor data and a gait waveform obtained by interpolating data of the missing section by the interpolation device. The gait measurement device measures the gait of the user based on the gait event detected from the acquired gait waveform. The gait measurement device outputs information regarding the measured gait of the user.

In the method according to the present example embodiment, focusing on the periodicity of gait, interpolation data for interpolating a missing section of data generated in time-series data of sensor data measured according to the gait of the user is generated. In the method according to the present example embodiment, interpolation data is generated using data of the same gait phase of the gait waveforms of different gait cycles. Therefore, according to the method according to the present example embodiment, since the interpolation data generated focusing on the periodicity of gait is used, the data of the missing section can be interpolated including the feature of the missing section of the data included in the time-series data of the sensor data.

When sensor data measured by a wearable sensor having a wireless communication function is communicated, data missing may occur due to a communication failure. Data missing that can occur due to communication failure is often continuous rather than discrete. For example, from the time-series data of the sensor data, the entire important data used for feature extraction and the like may be lost. According to the method of the present example embodiment, since the missing section of the data is interpolated using the similarity and periodicity of the gait motion, the data of the long missing section over the gait phase of several % to more than tens % can be interpolated. The method according to the present example embodiment can also be used for removal of an outlier measured due to a circuit factor and leveling of an abnormal value measured due to an unknown cause.

In one aspect of the present example embodiment, the gait information processing unit cuts out a waveform corresponding to one gait cycle from the time-series data. The gait information processing unit normalizes a gait phase included in the cut-out waveform to generate a gait waveform. According to the present aspect, since the gait phases of the plurality of gait waveforms generated from the time-series data of the sensor data are unified, it is easy to interpolate data between the plurality of gait waveforms.

In one aspect of the present example embodiment, regarding the missing section included in the time-series data, the interpolation unit selects at least one piece of data of the gait phase included in the missing section from at least one gait waveform in which the data of the gait phase included in the missing section is not missing. The interpolation unit generates interpolation data using the data of the selected gait phase. According to the present aspect, the missing section can be interpolated using data of another gait cycle.

In one aspect of the present example embodiment, the interpolation unit calculates an average value of data of the gait phase selected from the gait waveforms of the plurality of gait cycles. The interpolation unit generates interpolation data by using the calculated average value of the data of the gait phase. According to the present aspect, since the data of the missing section is interpolated based on the average value of the data of the plurality of gait cycles, it is possible to generate interpolation data in which the feature of gait is more reflected.

In one aspect of the present example embodiment, the interpolation unit generates interpolation data by an analytical method for a gait period in which data variation is small. The interpolation unit generates interpolation data using data of a gait cycle different from the gait cycle including the missing section for a gait period in which data variation is large. According to the present aspect, interpolation data is generated by an analytical method for a gait period in which data variation is small, so that calculation time and a calculation amount can be reduced.

### Second Example Embodiment

Next, a gait measurement system according to a second example embodiment will be described with reference to the drawings. The gait measurement system according to the present example embodiment is different from that of the first example embodiment in that interpolation data of a missing section is generated based on a deviation value in an overall distribution of data values (amplitude values) for each gait phase regarding gait waveforms of a plurality of gait cycles.

### (Configuration)

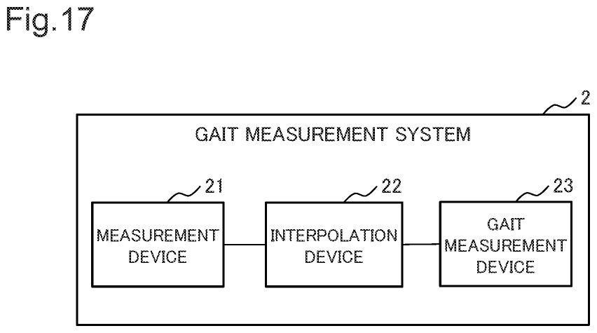

FIG. 17 is a block diagram illustrating a configuration of a gait measurement system 2 according to the present example embodiment. The gait measurement system 2 includes a measurement device 21, an interpolation device 22, and a gait measurement device 23. The interpolation device 22 may be connected to the measurement device 21 and the gait measurement device 23 in a wired manner or in a wireless manner. The measurement device 21, the interpolation device 22, and the gait measurement device 23 may be configured by a single device. The gait measurement system 2 may include the interpolation device 22 and the gait measurement device 23 except for the measurement device 21.

The measurement device 21 has a configuration similar to that of the measurement device 11 according to the first example embodiment. The measurement device 21 is installed on the foot portion. The measurement device 21 measures acceleration measured by the acceleration sensor (also referred to as spatial acceleration) and an angular velocity measured by the angular velocity sensor (also referred to as spatial angular velocity) as physical quantities regarding the movement of the foot of the user wearing the footwear. The physical quantity regarding the movement of the foot measured by the measurement device 21 includes a speed, an angle, and a position (trajectory) calculated by integrating the acceleration and the angular velocity. The measurement device 21 converts the measured physical quantity into digital data (also referred to as sensor data). The measurement device 21 transmits the converted sensor data to the interpolation device 22.

The interpolation device 22 receives sensor data from the measurement device 21. The interpolation device 22 generates time-series data of the received sensor data. The interpolation device 22 generates time-series data based on the time stamp included in the sensor data. The interpolation device 22 cuts out a waveform for one gait cycle from the generated time-series data. For example, the interpolation device 22 extracts a waveform corresponding to one gait cycle with the timing of heel strike or heel rise as a start point. The interpolation device 22 normalizes the cut waveform for one gait cycle to generate a waveform (also referred to as a gait waveform) for one gait cycle.

The interpolation device 22 specifies a portion of the time-series data of the sensor data in which the number of the time stamp is missing as a missing section. The interpolation device 22 generates interpolation data for interpolating the data of the missing section based on the overall distribution of the data values of the gait phase included in the missing section. The overall distribution is a distribution of data values for each gait phase included in the missing section, in which a gait waveform of a plurality of gait cycles used for interpolation of the missing section is the overall distribution. In other words, the interpolation device 22 calculates the statistic in the overall distribution of the data values of the gait phase relevant to the missing section by using the data values (amplitude values) of the gait waveforms of the plurality of gait cycles having no missing in the gait phase of the missing section. The interpolation device 22 generates interpolation data of the missing section based on the statistic in the overall distribution of the data values for each gait phase included in the missing section. In particular, the interpolation device 22 generates the interpolation data of the missing section based on the representative value in the overall distribution of the data values for each gait phase included in the missing section. For example, the interpolation device 22 generates the interpolation data of the missing section based on the deviation value in the overall distribution of the data value (amplitude value) for each gait phase included in the missing section. For example, the interpolation device 22 may generate the interpolation data of the missing section based on the standard deviation, the variance, the deviation, or the like instead of the deviation value. The interpolation device 22 interpolates the missing section using the generated interpolation data.

The gait measurement device 23 has a configuration similar to that of the gait measurement device 13 according to the first example embodiment. The gait measurement device 23 acquires a gait waveform not including the missing section from the interpolation device 22. The gait measurement device 23 executes measurement regarding the gait using the acquired gait waveform. The gait measurement device 23 outputs a measurement result (also referred to as gait information) regarding the gait.

For example, the interpolation device 22 and the gait measurement device 23 are mounted on a server (not illustrated) or the like. For example, the interpolation device 22 and the gait measurement device 23 may be achieved by an application server. For example, the interpolation device 22 and the gait measurement device 23 may be achieved by application software or the like installed in a mobile terminal (not illustrated).

### [Interpolation Device]

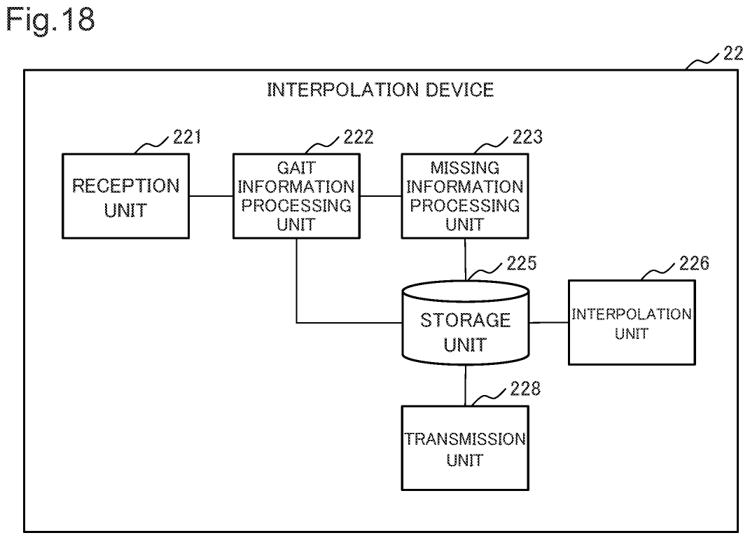

Next, the detailed configuration of the interpolation device 22 will be described with reference to the drawings. FIG. 18 is a block diagram illustrating an example of the detailed configuration of the interpolation device 22. The interpolation device 22 includes a reception unit 221, a gait information processing unit 222, a missing information processing unit 223, a storage unit 225, an interpolation unit 226, and a transmission unit 228.

The reception unit 221 has a configuration similar to that of the reception unit 121 according to the first example embodiment. The reception unit 221 receives sensor data from a measurement device (not illustrated). The reception unit 221 outputs the received sensor data to the gait information processing unit 222.

The gait information processing unit 222 has a configuration similar to that of the gait information processing unit 122 according to the first example embodiment. The gait information processing unit 222 acquires sensor data from the reception unit 221. The gait information processing unit 222 generates time-series data of the acquired sensor data. The gait information processing unit 222 generates time-series data based on the time stamp included in the sensor data. The gait information processing unit 222 cuts out a waveform for one gait cycle from the generated time-series data. The gait information processing unit 222 normalizes the cut-out waveform for one gait cycle and generates a gait waveform for one gait cycle. The gait information processing unit 222 stores the normalized gait waveform in the storage unit 225.

The missing information processing unit 223 has a configuration similar to that of the missing information processing unit 123 according to the first example embodiment. The missing information processing unit 223 acquires a missing waveform. The missing information processing unit 223 stores information regarding the missing section included in the missing waveform (also referred to as missing information) in the storage unit 225 in association with the missing waveform.

The storage unit 225 has a configuration similar to that of the storage unit 125 according to the first example embodiment. The gait waveform is stored in the storage unit 225. The gait waveform stored in the storage unit 225 includes a gait waveform not including the missing section (also referred to as a normal waveform), a gait waveform including the missing section (also referred to as a missing waveform), and a gait waveform in which the missing section is repaired (also referred to as a restoration waveform). The missing waveform is associated with missing information of the missing waveform. The gait waveform stored in the storage unit 225 is acquired by the transmission unit 228 at a predetermined timing and transmitted from the transmission unit 228 to the gait measurement device (not illustrated).

The interpolation unit 226 acquires, from the storage unit 225, a plurality of gait waveforms having no missing in the gait phase of the missing section included in the missing waveform of the interpolation target. The interpolation unit 226 calculates a statistic in the overall distribution of the data values of the gait phase relevant to the missing section in the data of the plurality of gait waveforms using the acquired data values (amplitude values) of the plurality of gait waveforms. In particular, the interpolation unit 226 calculates, as the statistic, a deviation value or a representative value such as a standard deviation, a variance, or a deviation. For example, the interpolation unit 226 calculates a deviation value of the data value of the gait phase included in the missing section in the overall distribution of the data of the plurality of gait waveforms. The interpolation unit 226 generates interpolation data based on the calculated statistic. For example, the interpolation unit 226 generates interpolation data based on the deviation value. For example, the interpolation unit 226 may generate the interpolation data of the missing section based on the standard deviation, the variance, the deviation, or the like instead of the deviation value. The interpolation unit 226 interpolates the missing section using the generated interpolation data.

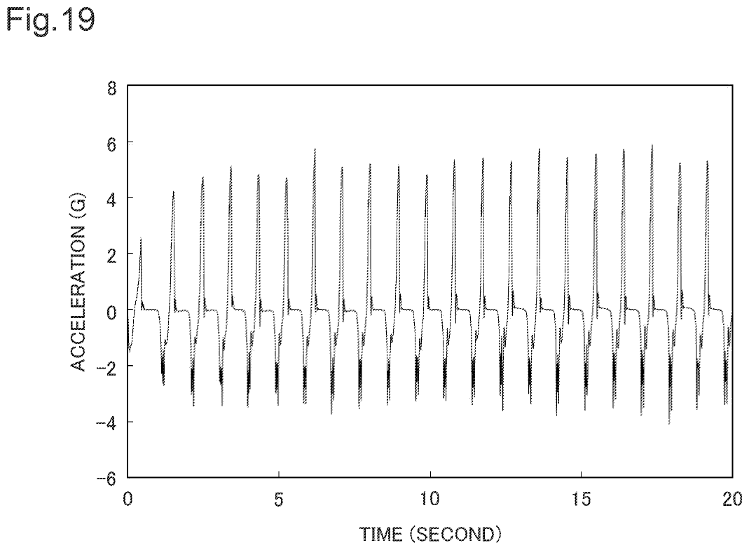

Here, a method of generating interpolation data based on a deviation value, which is one of statistics (representative values), will be described with reference to some drawings (FIGS. 19 to 23). FIG. 19 is time-series data of an acceleration in the traveling direction (Y-direction acceleration) measured along with gait of the user. The time-series data in FIG. 19 includes waveforms of a plurality of gait cycles in a measurement period of 20 seconds. The waveform of each gait cycle shows a similar pattern although there is a difference in data value (amplitude value).

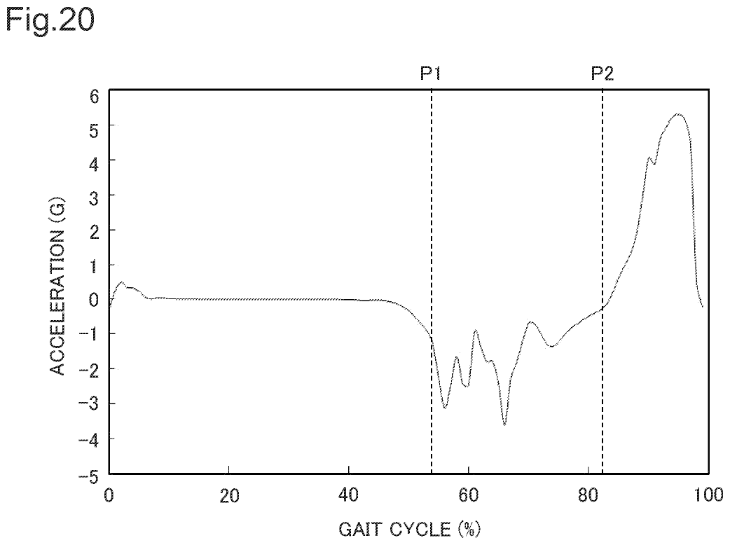

FIG. 20 is a waveform (gait waveform) obtained by cutting out one of waveforms of a plurality of gait cycles included in the time-series data of FIG. 19 and normalizing the cut-out waveform. In the gait waveform of FIG. 20, a pattern including feature of gait is seen. FIG. 20 illustrates the gait phases (P1, P2) used in the following description. Hereinafter, the gait phase P1 and the gait phase P2 are applied not only to the gait cycle of FIG. 20 but also to a plurality of gait cycles.

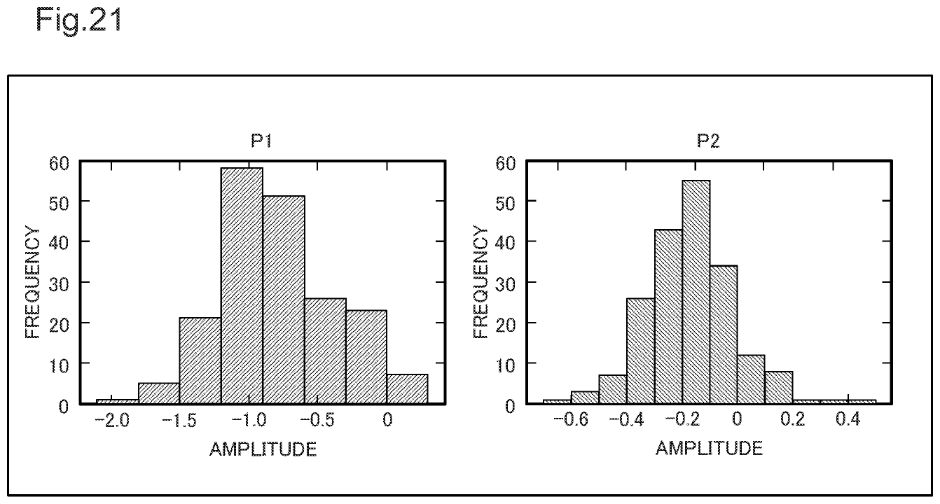

FIG. 21 is a frequency distribution of data values (amplitude values) in the gait phase P1 and the gait phase P2 in which the gait waveforms of a plurality of gait cycles cut out from the time-series data of FIG. 19 are set as samples (whole). For the gait phase P1, the frequency distribution of the data values shows a distribution close to the normal distribution. Also in the gait phase P2, the frequency distribution of the data values shows a distribution close to the normal distribution. Also for all the other gait phases, when the frequency distribution of data values for every 1% is normalized and discriminated by the Kolmogorov-Smirnov test, a positive result is obtained. That is, the frequency distribution of the data values in each gait phase of the gait waveforms of the plurality of gait cycles shows a distribution close to the normal distribution regardless of the gait cycle. This indicates that the interpolation data can be estimated based on the overall distribution of the data values for each gait phase with respect to the gait waveforms of the plurality of gait cycles. In other words, the interpolation data can be estimated based on the statistic in the overall distribution of the data values for each gait phase with respect to the gait waveforms of the plurality of gait cycles.

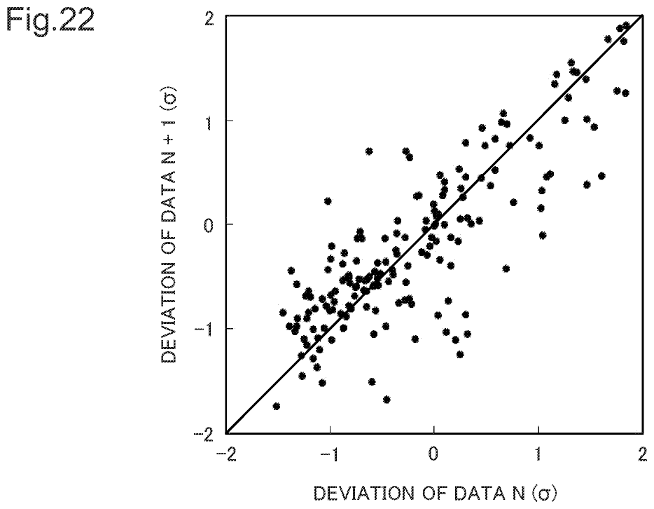

FIG. 22 is a graph illustrating a correlation of deviation values in an overall distribution of data values of the data N and the data N+1 of the continuous gait phase in the time-series data of the sensor data of FIG. 19 (N is a natural number). As illustrated in FIG. 22, the deviation value between consecutive data N and data N+1 shows a substantially proportional relationship. That is, the deviation value in the overall distribution of the data value in the gait phase P2 indicates a similar value for the consecutive data N and data N+1.

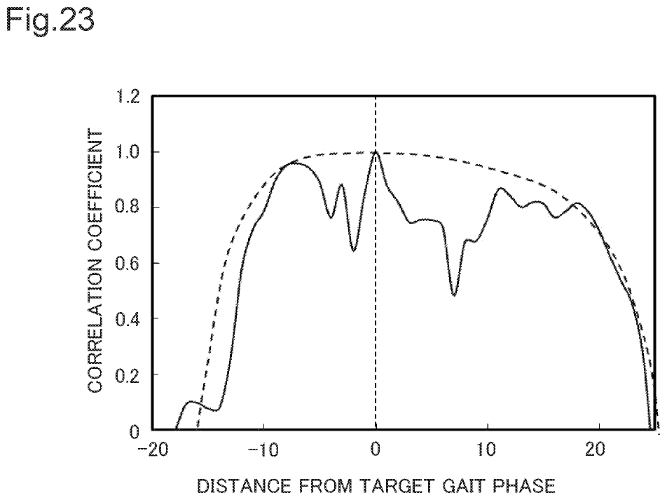

FIG. 23 is a graph illustrating a correlation coefficient of a deviation value according to a distance from a target gait phase (gait phase P2). A solid line in FIG. 23 indicates a correlation coefficient between a deviation value of a data value of the gait phase P2 and a data value of a gait phase different from the gait phase P2 regarding samples of a gait waveform of a plurality of gait cycles cut out from the time-series data in FIG. 19. A broken line in FIG. 23 is a curve obtained by fitting a correlation coefficient (solid line) to a polynomial. As illustrated in FIG. 23, it can be seen that the correlation coefficient of the deviation value is larger and the deviation value indicates a similar value as the gait phase is in a closer distance from the target gait phase. In other words, as the gait phase is in a closer distance from the target gait phase, the correlation coefficient is larger and the value of the deviation value is closer. That is, the interpolation data of the target gait phase can be generated using the data value of the gait phase having the deviation value close to the deviation value of the target gait phase.

For example, the interpolation unit 226 calculates interpolation data by calculating back from the calculated deviation value of the gait phase, and generates interpolation data for interpolating the missing section. For example, the interpolation unit 226 may generate interpolation data based on a representative value such as a standard deviation, a variance, or a deviation instead of the deviation value. For example, the interpolation unit 226 calculates a value dⱼ of the interpolation data in a gait phase j by calculating the following Expression 1 back. The following Expression 1 is a calculation expression of a deviation value Tⱼ of the data value of the gait phase j included in the missing section.

$${T}_{j} = \frac{10 \times {( {x}_{j} - μ )}^{2}}{σ} + 50 \qquad ( 1 )$$

In the above Expression 1, μ is an average value of data values of the gait phase j in a plurality of gait waveforms included in the target data. σ is a standard deviation of the data value of the gait phase j in the plurality of gait waveforms included in the target data. In a case where interpolation data is generated based on a representative value such as a standard deviation, a variance, or a deviation instead of the deviation value, a calculation expression such as the standard deviation, the variance, or the deviation may be back-calculated to calculate a value of the interpolation data.

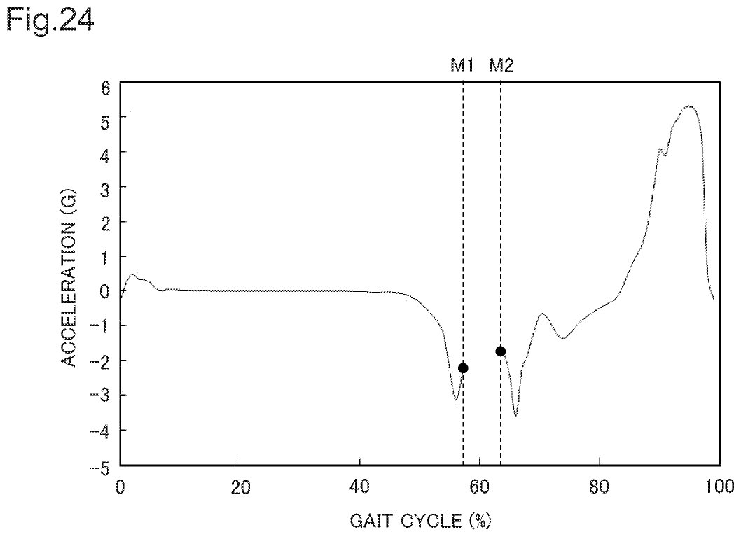

For example, the interpolation unit 226 may estimate the interpolation data of the missing section based on the deviation values of the data values of the start point and the end point of the missing section included in the missing waveform of the interpolation target. FIG. 24 is a graph for describing an example of interpolation of the missing section based on the deviation values of the start point (gait phase M1) and the end point (gait phase M2) of the missing section included in the missing waveform of the interpolation target. For example, the interpolation unit 226 calculates, for the missing section, an average value of the deviation value in the overall distribution of the data values of the gait phase M1 and the deviation value in the overall distribution of the data values of the gait phase M2. The interpolation unit 226 estimates interpolation data from the overall distribution of the data values by using the average value of the deviation values of the data values of the gait phase M1 and the gait phase M2. In the method of FIG. 24, as the distance between the gait phase M1 and the gait phase M2 of the missing section is closer, the estimation accuracy of the interpolation data is higher.

For example, the method of generating interpolation data by the interpolation unit 226 according to the present example embodiment may be combined with the method of generating interpolation data by the interpolation unit 126 according to the first example embodiment. For example, interpolation data may be generated by the method according to the first example embodiment, and the start point and the end point of the generated interpolation data may be corrected by the method of the present example embodiment.

The interpolation unit 226 stores the gait waveform (restoration waveform) in which the missing section is interpolated in the storage unit 225. For example, the interpolation unit 226 may attach a label to the missing section so that the repaired missing section can be specified in the restoration waveform. For example, the interpolation unit 226 may cause the storage unit 225 to store a restoration waveform (also referred to as a temporary restoration waveform) in which temporary interpolation data is inserted into the missing section. The restoration waveform stored in the storage unit 225 is used for gait measurement by a gait measurement device (not illustrated), similarly to other gait waveforms.

The transmission unit 228 acquires the gait waveform from the storage unit 225. The transmission unit 228 acquires the gait waveform including the normal waveform and the restoration waveform from the storage unit 225. The transmission unit 228 transmits the acquired gait waveform to a gait measurement device (not illustrated). The transmission unit 228 may transmit the gait waveform to the gait measurement device via a wire such as a cable, or may transmit the gait waveform to the gait measurement device via wireless communication. A communication system between the transmission unit 228 and the gait measurement device is not particularly limited.

### (Operation)

Next, the operation of the interpolation device 22 will be described with reference to the drawings. Hereinafter, an example in which the interpolation unit 226 included in the interpolation device 22 interpolates the missing section based on the deviation values of the start point and the end point of the missing section will be described. The following operation of the interpolation device 22 may include processing and order different from those of the interpolation unit 226 described above. Since the processing of the gait information processing unit 222 and the missing information processing unit 223 is similar to that of the first example embodiment, it is omitted.

### [Interpolation Processing]

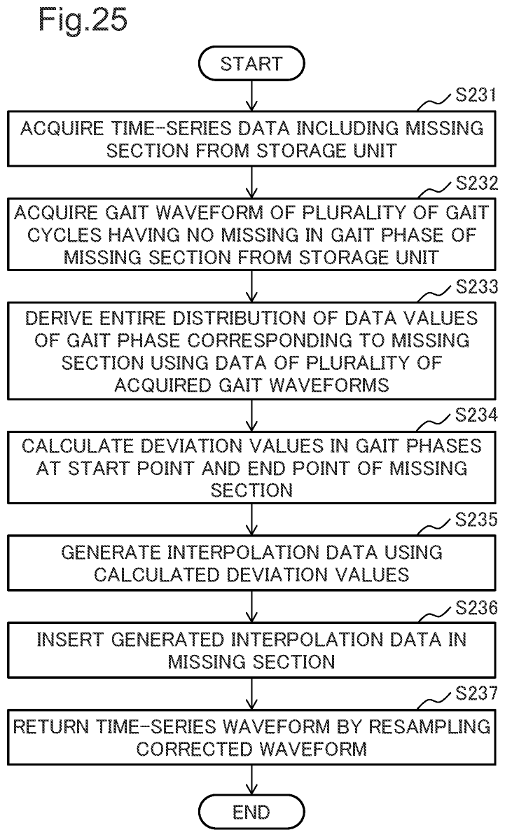

The interpolation processing by the interpolation unit 226 will be described with reference to a flowchart. FIG. 25 is a flowchart for explaining the interpolation processing of the interpolation unit 226. In the description along the flowchart of FIG. 25, the interpolation unit 226 will be described as an operation subject.

In FIG. 25, first, the interpolation unit 226 acquires time-series data (missing waveform) including the missing section from the storage unit 225 (step S231). The interpolation unit 226 may acquire the missing waveform from the gait information processing unit 222. The missing waveform is associated with missing information indicating the gait phase of the missing section.

Next, the interpolation unit 226 acquires the gait waveforms of the plurality of gait cycles having no missing in the gait phase of the missing section included in the missing waveform from the storage unit 225 (step S232).

Next, the interpolation unit 226 derives the overall distribution of the data of the gait phase relevant to the missing section using the acquired data of the plurality of gait waveforms (step S233).

Next, the interpolation unit 226 calculates deviation values of the gait phases at the start point and the end point of the missing section in the overall distribution of the data values of the gait phase included in the missing section (step S234).

Next, the interpolation unit 226 generates interpolation data of the missing section using the calculated deviation value (step S235).

Next, the interpolation unit 226 inserts the generated interpolation data into the missing section (step S236). For example, the interpolation unit 226 corrects the start point and the end point of the interpolation data, and inserts the interpolation data into the missing section.

Next, the interpolation unit 226 resamples the corrected waveform and returns the corrected waveform to the time-series data (step S237). Step S237 is performed because the original time-series data is required when the gait speed, the stride, and the like are calculated. When the original time-series data is not required, step S237 may be omitted.

As described above, the gait measurement system according to the present example embodiment includes the measurement device, the interpolation device, and the gait measurement device. The measurement device is disposed on the user's footwear. The measurement device measures the spatial acceleration and the spatial angular velocity according to the gait of the user. The measurement device generates sensor data based on the measured spatial acceleration and spatial angular velocity. The measurement device outputs the generated sensor data to the interpolation device. The interpolation device includes a reception unit, a gait information processing unit, a missing information processing unit, a storage unit, an interpolation unit, and a transmission unit. The reception unit receives the sensor data transmitted from the measurement device. The gait information processing unit generates time-series data of the sensor data. The gait information processing unit generates a gait waveform for each gait cycle using the time-series data of the sensor data regarding the movement of the foot, and specifies a missing section of the data in the time-series data. The gait information processing unit stores the generated gait waveform and information regarding the specified missing section in the storage unit. The storage unit stores a gait waveform, information regarding a missing section, and a restoration waveform obtained by interpolating the missing section. The missing information processing unit calculates a gait phase of the specified missing section. The interpolation unit generates interpolation data of the missing section based on a representative value of data for each gait phase included in the missing section in the data of the gait waveforms of a plurality of gait cycles used for interpolation of the missing section. For example, the interpolation unit generates interpolation data of the missing section by using a deviation value of data for each gait phase included in the missing section. The interpolation unit interpolates the generated interpolation data in the missing section. The gait measurement device acquires, from the interpolation device, a gait waveform not including the missing section generated by the interpolation device using the time-series data of the sensor data and a gait waveform obtained by interpolating data of the missing section by the interpolation device. The gait measurement device measures the gait of the user based on the gait event detected from the acquired gait waveform. The gait measurement device outputs information regarding the measured gait of the user.

In the method according to the present example embodiment, focusing on the periodicity of gait, interpolation data for interpolating a missing section of data generated in time-series data of sensor data measured according to the gait of the user is generated. In the method according to the present example embodiment, interpolation data is generated based on a representative value of data for each gait phase included in the missing section. In the method according to the present example embodiment, interpolation data is generated based on a distribution of data of the gait waveforms of a plurality of gait cycles generated from time-series data. According to the method of the present example embodiment, since the data of the gait waveform including the sudden abnormality is smoothed by generating the interpolation data based on the overall distribution, the data of the missing section can be restored with higher accuracy.

### Third Example Embodiment

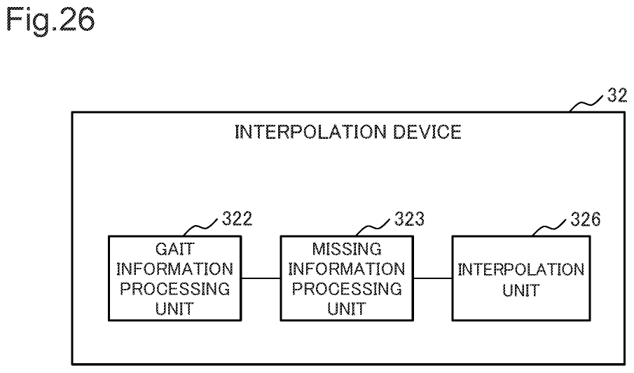

Next, an interpolation device according to a third example embodiment will be described with reference to the drawings. The interpolation device according to the present example embodiment has a configuration in which the interpolation devices according to the first and second example embodiments are simplified. FIG. 26 is a block diagram illustrating an example of a configuration of an interpolation device 32 according to the present example embodiment. The interpolation device 32 includes a gait information processing unit 322, a missing information processing unit 323, and an interpolation unit 326.

The gait information processing unit 322 generates a gait waveform for each gait cycle using the time-series data of the sensor data regarding the movement of the foot, and specifies a missing section of the data in the time-series data. The missing information processing unit 323 calculates a gait phase of the specified missing section. The interpolation unit 326 generates interpolation data for interpolating the missing section by using the data of the gait phase of the missing section in the gait waveform of the gait cycle different from the gait cycle including the missing section. The interpolation unit 326 interpolates the generated interpolation data into the missing section.

According to the present example embodiment, since the interpolation data generated focusing on the periodicity of gait is used, it is possible to interpolate the data of the missing section including the feature of the missing section of the data included in the time-series data of the sensor data.

### (Hardware)

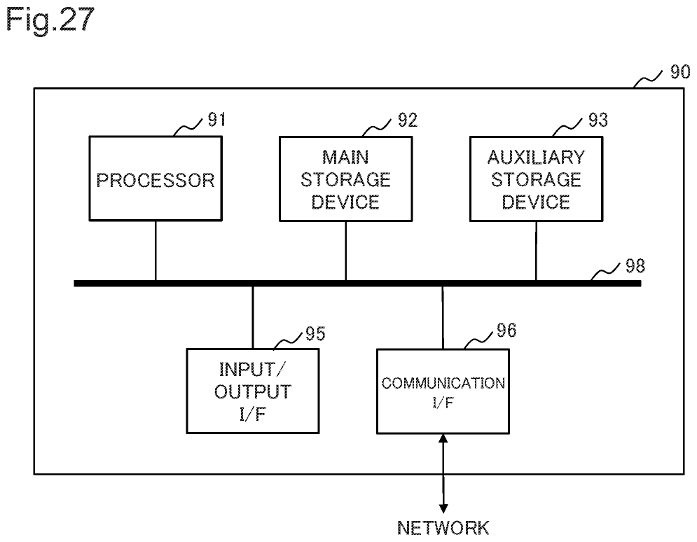

Here, a hardware configuration for executing processing of the control unit according to each example embodiment of the present disclosure will be described using an information processing device 90 of FIG. 27 as an example. The information processing device 90 in FIG. 27 is a configuration example for executing the control and processing of each example embodiment, and does not limit the scope of the present disclosure.

As illustrated in FIG. 27, the information processing device 90 includes a processor 91, a main storage device 92, an auxiliary storage device 93, an input/output interface 95, and a communication interface 96. In FIG. 27, the interface is abbreviated as an I/F. The processor 91, the main storage device 92, the auxiliary storage device 93, the input/output interface 95, and the communication interface 96 are data-communicably connected to each other via a bus 98. The processor 91, the main storage device 92, the auxiliary storage device 93, and the input/output interface 95 are connected to a network such as the Internet or an intranet via the communication interface 96.

The processor 91 develops a program stored in the auxiliary storage device 93 or the like in the main storage device 92. The processor 91 executes the program developed in the main storage device 92. In the present example embodiment, a software program installed in the information processing device 90 may be used. The processor 91 executes processing or control according to the present example embodiment.

The main storage device 92 has an area in which a program is developed. A program stored in the auxiliary storage device 93 or the like is developed in the main storage device 92 by the processor 91. The main storage device 92 is implemented by, for example, a volatile memory such as a dynamic random access memory (DRAM). A nonvolatile memory such as a magnetoresistive random access memory (MRAM) may be configured and added as the main storage device 92.

The auxiliary storage device 93 stores various data such as programs. The auxiliary storage device 93 is implemented by a local disk such as a hard disk or a flash memory. Various data may be stored in the main storage device 92, and the auxiliary storage device 93 may be omitted.

The input/output interface 95 is an interface for connecting the information processing device 90 and a peripheral device. The communication interface 96 is an interface for connecting to an external system or device through a network such as the Internet or an intranet based on a standard or a specification. The input/output interface 95 and the communication interface 96 may be shared as an interface connected to an external device.

An input device such as a keyboard, a mouse, or a touch panel may be connected to the information processing device 90 as necessary. These input devices are used to input information and settings. When the touch panel is used as an input device, the display screen of the display device may also serve as the interface of the input device. Data communication between the processor 91 and the input device may be mediated by the input/output interface 95.

The information processing device 90 may be provided with a display device for displaying information. In a case where a display device is provided, the information processing device 90 may include a display control device (not illustrated) for controlling display of the display device. The display device may be connected to the information processing device 90 via the input/output interface 95.

The information processing device 90 may be provided with a drive device. The drive device mediates reading of data and a program from a recording medium, writing of a processing result of the information processing device 90 to the recording medium, and the like between the processor 91 and the recording medium (program recording medium). The drive device may be connected to the information processing device 90 via the input/output interface 95.

The above is an example of the hardware configuration for enabling the control and processing according to each example embodiment of the present invention. The hardware configuration of FIG. 27 is an example of a hardware configuration for executing the control and processing of each example embodiment, and does not limit the scope of the present invention. A program for causing a computer to execute the control and processing according to each example embodiment is also included in the scope of the present invention. Further, a program recording medium in which the program according to each example embodiment is recorded is also included in the scope of the present invention. The recording medium can be achieved by, for example, an optical recording medium such as a compact disc (CD) or a digital versatile disc (DVD). The recording medium may be implemented by a semiconductor recording medium such as a universal serial bus (USB) memory or a secure digital (SD) card. The recording medium may be implemented by a magnetic recording medium such as a flexible disk, or another recording medium. When a program executed by the processor is recorded in a recording medium, the recording medium is associated to a program recording medium.

The components of each example embodiment may be arbitrarily combined. The components of each example embodiment may be implemented by software or may be implemented by a circuit.

Although the present invention has been described with reference to the example embodiments, the present invention is not limited to the above example embodiments. Various modifications that can be understood by those skilled in the art can be made to the configuration and details of the present invention within the scope of the present invention.

### REFERENCE SIGNS LIST

1, 2 gait measurement system
11, 21 measurement device
12, 22, 32 interpolation device
13, 23 gait measurement device
111 acceleration sensor
112 angular velocity sensor
113 control unit
115 transmission unit
121, 221 reception unit
122, 222, 322 gait information processing unit
123, 223, 323 missing information processing unit
125, 225 storage unit
126, 226, 326 interpolation unit
128, 228 transmission unit
131 acquisition unit
132 detection unit
133 gait measuring unit

What is claimed is:

1. An interpolation device comprising:
a first memory storing instructions; and
a first processor connected to the first memory and configured to execute the instructions to:
generate a gait waveform for each gait cycle by using time-series data of sensor data regarding a movement of a foot and specify a missing section of data in the time-series data;
calculate a gait phase of the specified missing section; and
generate interpolation data for interpolating the missing section by using data of the gait phase of the missing section in a gait waveform with a gait cycle different from a gait cycle including the missing section, and interpolate the generated interpolation data to the missing section.

2. The interpolation device according to claim 1, wherein
the first processor is configured to execute the instructions to
cut out a waveform corresponding to one gait cycle from the time-series data, and
generate the gait waveform by normalizing the gait phase included in the cut-out waveform.

3. The interpolation device according to claim 1, wherein
the first processor is configured to execute the instructions to
select, for the missing section included in the time-series data, at least one piece of data of the gait phase included in the missing section from at least one of the gait waveforms in which data of the gait phase included in the missing section is not missing, and
generate the interpolation data using data of the selected gait phase.

4. The interpolation device according to claim 3, wherein
the first processor is configured to execute the instructions to
calculate an average value of data of the gait phase selected from the gait waveforms of a plurality of the gait cycles, and
generate the interpolation data using an average value of calculated data of the gait phase.

5. The interpolation device according to claim 1, wherein
the first processor is configured to execute the instructions to
generate the interpolation data by an analytical method for a gait period in which data variation is small, and
generate the interpolation data using data of the gait cycle different from the gait cycle including the missing section for the gait period in which data variation is large.

6. The interpolation device according to claim 1, wherein
the first processor is configured to execute the instructions to
generate the interpolation data of the missing section based on a representative value of data for each of the gait phases included in the missing section in data of the gait waveforms of a plurality of the gait cycles used for interpolation of the missing section.

7. The interpolation device according to claim 6, wherein
the first processor is configured to execute the instructions to
generate the interpolation data of the missing section using a deviation value of data for each of the gait phases included in the missing section.

8. A gait measurement system comprising:
an interpolation device according to claim 1;
a measurement device that is disposed on footwear of a user, measures a spatial acceleration and a spatial angular velocity according to gait of the user, generates sensor data based on the measured spatial acceleration and the measured spatial angular velocity, and outputs the generated sensor data to the interpolation device; and
a gait measurement device comprising
a second memory storing instructions; and
a second processor connected to the second memory and configured to execute the instructions to
acquire, from the interpolation device, a gait waveform not including a missing section generated by the interpolation device using time-series data of the sensor data and a gait waveform obtained by interpolating data of a missing section by the interpolation device,
measure a gait of the user based on a gait event detected from the acquired gait waveform, and
output information regarding the measured gait of the user.

9. An interpolation method causing a computer to execute:
generating a gait waveform for each gait cycle by using time-series data of sensor data regarding a movement of a foot;
specifying a missing section of data in the time-series data;
calculating a gait phase of the specified missing section;
generating interpolation data for interpolating the missing section by using data of the gait phase of the missing section in a gait waveform having a gait cycle different from a gait cycle including the missing section; and
interpolating the generated interpolation data into the missing section.

10. A non-volatile recording medium having stored therein a program causing a computer to execute:
a processing of generating a gait waveform for each gait cycle by using time-series data of sensor data regarding a movement of a foot;
a processing of specifying a missing section of data in the time-series data;
a processing of calculating a gait phase of the specified missing section;
a processing of generating interpolation data for interpolating the missing section by using data of the gait phase of the missing section in a gait waveform having a gait cycle different from a gait cycle including the missing section; and
a processing of interpolating the generated interpolation data into the missing section.
